(19) United States
(12) Patent Application Publication
(10) Pub. No.: US 2024/0368280 A1
(43) Pub. Date: Nov. 7, 2024

(54) ANTI-CECAM6 ANTIBODIES WITH REDUCED SIDE-EFFECTS

(71) Applicant: Deutsches Krebsforschungszentrum Stiftung des öffentlichen Rechts, Heidelberg (DE)

(72) Inventors: Mark Trautwein, Wülfrath (DE); Jörg Willuda, Glienicke/Nordbahn (DE); Wolf-Dietrich Döcke, Berlin (DE); Pascale Buchmann, Leverkusen (DE); Rafael Carretero, Heidelberg (DE); Philipp Ellinger, Solingen (DE); Rienk Offringa, Heidelberg (DE); Franz Hendrik Nogai, Binningen (CH); Pedro Paz, Hercules, CA (US)

(21) Appl. No.: 18/688,693

(22) PCT Filed: Sep. 1, 2022
(86) PCT No.: PCT/EP2022/074394

§ 371 Date: Mar. 1, 2024

Related U.S. Application Data
(60) Provisional application No. 63/240,134, filed on Sep. 2, 2021.

(51) Int. Cl. C07K 16/28; A61P 35/00

(52) U.S. Cl. CPC C07K 16/2803; A61P 35/00; C07K 16/2818; C07K 16/2827; C07K 2317/21; C07K 2317/24; C07K 2317/92

(57) ABSTRACT

The present invention provides antibodies, that bind to human CEACAM6 and are able to relieve CEACAM6-mediated immunosuppression, wherein said antibodies have reduced side-effects during treatment. The present invention further provides isolated nucleic acids encoding said antibodies and vectors comprising same, isolated cells expressing said antibodies, methods of producing said antibodies and pharmaceutical compositions and kits comprising said antibodies. Antibodies according to the present invention can be used to treat cancer and might be used to treat other disorders and conditions associated with the expression of the CEACAM6.

### FIELD OF THE INVENTION

The present invention provides antibodies, that bind to human CEACAM6 and are able to relieve CEACAM6-mediated immunosuppression, wherein said antibodies have reduced side-effects during treatment. The present invention further provides isolated nucleic acids encoding said antibodies and vectors comprising same, isolated cells expressing said antibodies, methods of producing said antibodies and pharmaceutical compositions and kits comprising said antibodies. Antibodies according to the present invention can be used to treat cancer and might be used to treat other disorders and conditions associated with the expression of the CEACAM6.

### BACKGROUND OF THE INVENTION

### Prior Art

Several cancer types have the capacity to block effector functions of T-cells, limiting the efficacy of cancer immunotherapy. However, antibody blockade of immune checkpoint molecules is a clinically validated approach to reactivate the immune cells. The most prominent example is the blockade of the programmed cell death protein 1/programmed death ligand 1 (PD-1/PD-L1) axis. Several drugs are approved or currently under clinical development targeting this axis and impressive clinical responses have been reported in diseases such as melanoma, renal cell carcinoma, and lung cancer. Despite the success of these approaches, groups of patients either do not respond to the PD-1/PD-L1 inhibitors or they develop resistance to them and thus, novel immunotherapy solutions are needed.

CEACAM6 (carcinoembryonic antigen related cell adhesion molecule 6, also known as CD66c, non-specific cross-reacting antigen, NCA, or NCA 50/90) is an attractive target for therapeutic intervention in cancer immunotherapy. In humans, CEACAM6 is expressed on cells of several cancer types. The highest prevalence of membrane localized CEACAM6 expression is found in adenocarcinoma of the lung, colon, pancreas, and stomach, in which it was found to correlate with tumor progression and adverse clinical outcome. In addition, tumor infiltrating myeloid cells, especially granulocytes and, to a lesser degree, macrophages, express high levels of CEACAM6. In normal conditions, CEACAM6 is expressed on myeloid cells in blood with the highest levels seen on granulocytes, resident myeloid cells, and epithelial cells in the lung and intestine. While CEACAM6 orthologs exist in human and non-human primates, it has no known ortholog in rodents.

It has been demonstrated that the blockade of CEACAM6 by a monoclonal antibody (mAb) or silencing via small interfering ribonucleic acid (siRNA) reinstates T-cell activity against malignant plasma cells derived from multiple myelomas as well as other solid cancers (Witzens-Harig et al., Blood 2013 May 30; 121 (22): 4493-503; WO 2016/150899 A2). This suggests that CEACAM6, expressed on the surface of malignant cells, plays a role in the regulation of antitumor responses mediated by CD8-positive T-cells, which is consistent with the fact that CEACAM6 acts as an immunosuppressive factor in solid cancers.

Several anti-CEACAM6 antibodies exist. Most of them are non-human reagent antibodies, many of them are polyclonal. The specificity and selectivity to human CEACAM6 as well as cross-reactivity to monkey CEACAM6 is in most of the cases not disclosed or known. Therapeutic antibodies directed against CEACAM6 are also known in the art. Some are not selective to human CEACAM6 (e.g. MN-3 from Immunomedics, Neo201/h16C3 from Neogenix; both binding in addition to human CEACAM5). A single domain antibody 2A3 and its fusion variants (WO 2012/040824 A1 and Niu et al., J Control Release. 2012 Jul. 10; 161 (1): 18-24) are not characterized with respect to selectivity and cross-reactivity to monkey CEACAM6.

The murine antibody 9A6 (Genovac/Aldevron) was the first antibody described to be able to modulate the immunosuppressive activity of CEACAM6 (Witzens-Harig et al., Blood 2013 May 30; 121 (22): 4493-503). 9A6 inhibits the immunosuppressive activity of CEACAM6, leading to enhanced cytokine secretion by T cells in vitro and anti-tumor efficacy in vivo (Khandelwal et al., Poster Abstract 61, Meeting Abstract from 22nd Annual International Cancer Immunotherapy Symposium Oct. 6-8, 2014, New York City, USA). The murine antibody 9A6 does not exhibit cross-reactivity to monkey CEACAM6 (WO 2016/150899 A2). In addition, its murine nature precludes a direct therapeutic application in humans.

WO 2016/150899 A2 discloses a range of human anti-CEACAM6 antibodies, which are useful for therapeutic use, relieving the immunosuppressive activity of CEACAM6 which can be therapeutically applied in human cancer patients. These antibodies are specific for human and Macaca fascicularis CEACAM6 (Carcinoembryonic antigen-related cell adhesion molecule 6, CD66c, Non-specific cross-reacting antigen, NCA, NCA-50/90), and do not significantly cross-react with the closely related human CEACAM1, human CEACAM3, and human CEACAM5. Anti-CECAM6 antibody TPP-3310 disclosed in WO 2016/150899 A2 is a preferred embodiment of these antibodies.

Combination treatments of anti-CEACAM6 antibodies with other immunotherapeutic approaches were disclosed in WO 2020/099230 A1 (in combination with anti-PD1 and anti-PD-L1 antibodies) and in WO 2020/126808 A1 (in combination with anti-TIM3 antibodies).

It is known that clinical efficacy of many therapeutically applied antibodies is currently achieved only in subsets of patients. Thus, selection of the antibody isotype format represents an important step towards improved patient outcome (Vukovic et al., Clin Exp Immunol. 2021 March; 203(3):351-365). A plethora of Fc-engineering options exist to modulate effector function or half-life of natural antibody isotypes (Wang et al., Protein Cell. 2018 January; 9(1):63-73).

For instance, multiple mutational variants have been described that enhance CDC effector functions. Similarly, a multitude of mutations are known that enhance FcγR-dependent effector functions such as ADCC and ADCP. These enhancements cannot only be brought about by amino acid mutations but also by glycoengineering. A prominent example is the afucosylation of antibodies that is correlated with a stronger binding to FcγRIIIa and thus enhanced ADCC by NK cells.

For cases where mAbs are intended to engage cell surface receptors and prevent receptor-ligand interactions (i.e., antagonists), it may be desirable to reduce or eliminate effector function for example to prevent cell death of normal cells expressing the target or prevent unwanted cytokine secretion. It is recognized that the four human IgG subclasses, each have a different ability to elicit immune effector functions. For instance, IgG1 and IgG3 are able to recruit complement much more effectively than IgG2 and IgG4, while IgG2 and IgG4 have very limited ability to elicit ADCC. Fc engineering examples include human IgG4 variants L235E or F234A/L235A and the human IgG1 variant L234A/L235A (“LALA”; Xu et al., Cell Immunol 2000 Feb. 25; 200(1): 16-26). Another early approach intended to reduce effector function was to mutate the glycosylation site at N297 with mutations such as N297A, N297Q, and N297G (“aglycosylation”; Bolt et al., Eur J Immunol. 1993 February; 23(2): 403-11; Tao and Morrison, J Immunol. 1989 Oct. 15; 143(8): 2595-601; Walker et al., Biochem J. 1989 Apr. 15; 259(2): 347-53; Leabman et al., MAbs November-December 2013; 5(6): 896-903). Another variation is a cross-subclass approach to reduce effector function as exemplified by the approved anti-C5 therapeutic eculizumab, which carries CH1 and hinger region from IgG2 but carries CH2 and CH3 from IgG4. Other examples include L234F/L235E/P331S in human IgG1 (“FES”; Oganesyan et al., Acta Crystallogr D Biol Crystallogr. 2008 June; 64(Pt 6):700-4), P329G/L234A/L235A in human IgG1 (“PG-LALA”; Schlothauer et al., Protein Eng Des Sel 2016 October; 29(10):457-466), “IgG1sigma” (L234A/L235A/G237A/P238S/H268A/A330S/P331S, Tam et al., Antibodies (Basel) 2017 Sep. 1; 6(3): 12), and “IgG1-NNAS” (S298N/T299A/Y300S, Zhou et al., MAbs January-December 2020; 12(1): 1814583).

In addition, mutations have been described which increase the co-engagement of antigen and FcγRs through enhanced binding to e.g. FcγRIIb or FcγRIIa on FcγR-bearing antigen-positive cells.

Finally, addressing the interaction of Fc with FcRn allows to modulate the half-life of antibodies in vivo. Abrogating the interaction by e.g. H435A leads to an extremely short half-life, since the antibody is no longer protected from lysosomal degradation by FcRn recycling. In contrast, “YTE” (M252Y/S254T/T256E) and equivalent mutations have been shown to significantly extend the half-life by more efficient recycling from endosomes in both pre-clinical species as well as humans.

### Technical Problem

To study the therapeutic potential of the anti-CEACAM6 antibody TPP-3310 (disclosed in WO 2016/150899 A2) in cancer patients in a clinical trial, a human IgG2 format with reduced effector function was chosen, based on its favorable preclinical safety profile. Quite unexpectedly, neutropenia as an adverse effect occurred in in cancer patients treated with low doses of TPP-3310 (see Example 2).

So an antibody suitable for therapeutic use, which binds to human CEACAM6 and is able to relieve CEACAM6-mediated immunosuppression, wherein said antibody has reduced side-effects during treatment is highly desirable.

### Solution to Problem

As shown in this application, neutrophils can indeed surprisingly be activated by TPP-3310 in whole blood assays, recapitulating at least in part the clinical findings (see Example 3). However, this activation requires a very fine combined dependency of pre-stimulation, epitope and antibody isotype. Furthermore, the effect is Fc dependent and FcγRs are involved. This is completely unpredictable since even the strict dependency on an Fc part as well as the involvement of FcγRII would rather implicate human IgG1 as a very potent molecule.

In contrast to previous teachings, the inventors have found that for TPP-3310 (human IgG2) in fact changing the isotype to a human IgG1 completely prevents neutrophil activation in the whole blood assays. The considered to be more silent human IgG2 isotype is in fact a molecule capable of exerting the neutrophil activation effect (see Example 3).

On the other hand, human IgG1 format is precluded from use in a therapeutic antibody format just because of its strong interaction with FcγRs and thus strong and unwanted effector potential such as ADCC, ADCP and CDC activities.

Antibodies of the invention comprise an IgG1-based engineered format (L234A L235A in combination with aglycosylation, preferable N297A) without FcγR interaction and thus without effector function fulfilling the requirement of being incapable of effector function at the same time also incapable of activation of neutrophils in blood under pre-stimulated conditions.

Thus, neutropenia can be avoided as adverse event in therapeutic interventions in cancer patients with an anti-CEACAM6 IgG1-based engineered antibody (TPP-21518).

### SUMMARY OF THE INVENTION

The above-mentioned object and other objects are achieved by the teaching of the present invention.

### First Aspect of the Invention: An Anti-CECAM6 Antibody

In a first aspect, the present invention relates to an anti-CECAM6 antibody comprising an IgG1 Fc region lacking the glycans attached to the conserved N-linked site in the CH2 domains of the Fc region, wherein said IgG1 Fc region comprises at least the amino acid substitutions L234A and L235A, as numbered according to the EU index of Kabat.

In certain embodiments of the first aspect the invention provides an anti-CECAM6 antibody comprising an IgG1 Fc region, wherein said IgG1 Fc region comprises an amino acid substitution N297A, N297G, or N297Q as numbered according to the EU index of Kabat.

In certain embodiments of the first aspect the invention provides an anti-CECAM6 antibody comprising an IgG1 Fc region, wherein said IgG1 Fc region comprises an amino acid substitution N297A, N297G, or N297Q and at least the amino acid substitutions L234A and L235A, as numbered according to the EU index of Kabat.

In certain embodiments of the first aspect the invention provides an anti-CECAM6 antibody comprising an IgG1 Fc region, wherein said IgG1 Fc region comprises at least the amino acid substitutions N297A, L234A, and L235A, as numbered according to the EU index of Kabat.

In certain embodiments of the first aspect the invention provides an anti-CECAM6 antibody comprising an IgG1 Fc region, wherein said IgG1 Fc region comprises the amino acid substitutions N297A, L234A, and L235A, as numbered according to the EU index of Kabat.

In certain embodiments of the first aspect the anti-CECAM6 antibody mentioned supra competes for CEACAM6 binding with an antibody comprising a heavy chain variable region (VH) comprising the amino acid sequence of Seq ID No: 63 and a light chain variable region (VL) comprising the amino acid sequence of Seq ID No: 67.

In certain embodiments of the first aspect the anti-CECAM6 antibody mentioned supra comprises: a heavy chain variable region H-CDR1 comprising the amino acid sequence of SEQ ID NO: 64, a heavy chain variable region H-CDR2 comprising the amino acid sequence of SEQ ID NO: 65, a heavy chain variable region H-CDR3 comprising the amino acid sequence of SEQ ID NO: 66, a light chain variable region L-CDR1 comprising the amino acid sequence of SEQ ID NO: 68, a light chain variable region L-CDR2 comprising the amino acid sequence of SEQ ID NO: 69, and a light chain variable region L-CDR3 comprising the amino acid sequence of SEQ ID NO: 70.

In certain embodiments of the first aspect the anti-CECAM6 antibody mentioned supra comprises: a heavy chain variable region (VH) comprising the amino acid sequence of SEQ ID NO: 63, and a light chain variable region (VL) comprising the amino acid sequence of SEQ ID NO: 67.

In certain embodiments of the first aspect the anti-CECAM6 antibody mentioned supra comprises: a heavy chain (HC) comprising the amino acid sequence of SEQ ID NO: 71, and a light chain (LC) comprising the amino acid sequence of SEQ ID NO: 72.

In certain embodiments the invention provides an anti-CECAM6 antibody comprising a heavy chain (HC) comprising the amino acid sequence of SEQ ID NO: 71, and a light chain (LC) comprising the amino acid sequence of SEQ ID NO: 72.

### Further Aspects of the Invention

In a further aspect the invention provides a nucleic acid that encodes the anti-CECAM6 antibody of the first aspect, and a vector comprising said nucleic acid.

In a further aspect the invention provides an isolated cell expressing the anti-CECAM6 antibody of the first aspect. In preferred embodiments this cell is a prokaryotic or a eukaryotic cell.

In a further aspect the invention provides a method of producing the anti-CECAM6 antibody of the first aspect.

In a further aspect the invention provides an anti-CECAM6 antibody of the first aspect for use as a medicament, in particular for use as a medicament for the treatment of cancer. In certain embodiments of this aspect a method is provided for treating cancer associated with the undesired presence of CEACAM6, comprising administering to a subject in need thereof an effective amount of the anti-CECAM6 antibody of the first aspect.

In a further aspect the invention provides an anti-CEACAM6 antibody of the first aspect for use in simultaneous, separate, or sequential combination with an anti-PD-1 antibody or an anti-PD-L1 antibody in the treatment of cancer. In certain embodiments the anti-PD-1 antibody is nivolumab, or pembrolizumab, and the anti-PD-L1 antibody is atezolizumab, avelumab, or durvalumab. In certain embodiments of this aspect a method of treating cancer is provided comprising administering to a patient in need thereof an effective amount of an anti-CEACAM6 antibody of the first aspect in simultaneous, separate, or sequential combination with an anti-PD-1 antibody or an anti-PD-L1 antibody, preferably the anti-PD-1 antibody is nivolumab, or pembrolizumab, and the anti-PD-L1 antibody is atezolizumab, avelumab, or durvalumab.

In a further aspect the invention provides an anti-CEACAM6 antibody of the first aspect for use in simultaneous, separate, or sequential combination with an anti-TIM-3 antibody in the treatment of cancer. In certain embodiments the anti-TIM-3 antibody is cobolimab, MBG-453, BMS-986258, Sym-023, LY-3321367 or INCAGN-2390. In certain embodiments of this aspect a method of treating cancer is provided comprising administering to a patient in need thereof an effective amount of the anti-CEACAM6 antibody of the first aspect in simultaneous, separate, or sequential combination with an anti-TIM-3 antibody, preferably the anti-TIM-3 antibody is cobolimab, MBG-453, BMS-986258, Sym-023, LY-3321367 or INCAGN-2390.

In a further aspect the invention provides a pharmaceutical composition comprising the anti-CECAM6 antibody of the first aspect.

### DESCRIPTION OF THE FIGURES

FIG. 1: TNF-alpha plasma levels at different time points after start of the i.v. infusion of the anti-CEACAM6 antibody TPP-3310 to cancer patients. Three patients per dose cohort have been treated with either 2.5 mg, 5 mg, 10 mg or 30 mg of TPP-3310 in the clinical formulation over 1 hour. Mean values plus standard deviations are given. X-axis: Time after start of infusion in hours; Y-axis: Concentration of TNF-alpha in plasma [pg/mL]

FIG. 2: IL-6 plasma levels at different time points after start of the i.v. infusion of the anti-CEACAM6 antibody TPP-3310 to cancer patients. Three patients per dose cohort have been infused with either 2.5 mg, 5 mg, 10 mg or 30 mg of TPP-3310 in the clinical formulation over 1 hour. Mean values plus standard deviations are given. X-axis: Time after start of infusion in hours; Y-axis: Concentration of IL-6 in plasma [pg/mL]

FIG. 3: IL-10 plasma levels at different time points after start of the i.v. infusion of the anti-CEACAM6 antibody TPP-3310 to cancer patients. Three patients per dose cohort have been infused with either 2.5 mg, 5 mg, 10 mg or 30 mg of TPP-3310 in the clinical formulation over 1 hour. Mean values plus standard deviations are given. X-axis: Time after start of infusion in hours; Y-axis: Concentration of IL-10 in plasma [pg/mL]

FIG. 4: Plasma levels of myeloperoxidase (MPO) at different time points after start of the i.v. infusion of the anti-CEACAM6 antibody TPP-3310 to cancer patients. Three patients per dose cohort have been infused with either 2.5 mg, 5 mg, 10 mg or 30 mg of TPP-3310 in the clinical formulation over 1 hour. Relative values in comparison to pre-treatment are given as percentage mean values plus standard deviations. X-axis: Time after start of infusion in hours; Y-axis: Concentration of MPO in plasma [% vs. Oh pre-treatment levels]

FIG. 5. Neutrophil count of patients treated at 30 mg TPP-3310 at selected time points. A value of <0.5/nL is considered a severe neutrophil count decrease according to CTCAE criteria. N/A: Sample not taken. X-axis: Time after infusion. 1: pre dose; 2:24 hours; 3:48 hours; 4:7 days; 5:14 days; 6:21 days; Y-axis: Neutrophil count in/nL

FIG. 6: Myeloperoxidase (MPO) release with anti-CEACAM6 antibody TPP-3310 (human IgG2 format) (black columns) and corresponding isotype control antibody (white columns) with (+fMLP) and without (w/o fMLP) suboptimal fMLP stimulus. X-axis: concentration of antibody [μM]; Y-axis: pg/ml MPO.

FIG. 7: Myeloperoxidase (MPO) release with anti-CEACAM6 antibody TPP-5468 (human IgG1 format) (black columns) and corresponding isotype control antibody (white columns) with (+fMLP) and without (w/o fMLP) suboptimal fMLP stimulus. X-axis: concentration of antibody [μM]; Y-axis: pg/ml MPO.

FIG. 8: Myeloperoxidase (MPO) release with anti-CEACAM6 antibody 9A6 TPP-3470 (human IgG2 format) (black columns) and corresponding isotype control antibody (white columns) with (+fMLP) and without (w/o fMLP) suboptimal fMLP stimulus. X-axis: concentration of antibody [μM]; Y-axis: pg/ml MPO.

FIG. 9: Myeloperoxidase (MPO) release with anti-CEACAM6 antibody Neo201 TPP-1173 (human IgG1 format) (black columns) and corresponding isotype control antibody (white columns) with (+fMLP) and without (w/o fMLP) suboptimal fMLP stimulus. X-axis: concentration of antibody [μM]; Y-axis: pg/ml MPO.

FIG. 10: Myeloperoxidase (MPO) release with anti-CEACAM6 antibody Neo201 TPP-3688 (human IgG2 format) (black columns) and corresponding isotype control antibody (white columns) with (+fMLP) and without (w/o fMLP) suboptimal fMLP stimulus. X-axis: concentration of antibody [μM]; Y-axis: pg/ml MPO.

FIG. 11: Myeloperoxidase (MPO) release with anti-CEACAM6 antibody Fab fragment APP-1574 (derived from human IgG1) (black columns) and corresponding isotype control antibody fragment (white columns) with (+fMLP) and without (w/o fMLP) suboptimal fMLP stimulus. X-axis: concentration of antibody [μM]; Y-axis: pg/ml MPO.

FIG. 12: Myeloperoxidase (MPO) release with anti-CEACAM6 antibody F(ab)₂ fragment APP-6036 (derived from human IgG1) (black columns) and corresponding isotype control antibody fragment (white columns) with (+fMLP) and without (w/o fMLP) suboptimal fMLP stimulus. X-axis: concentration of antibody [μM]; Y-axis: pg/ml MPO.

FIG. 13: Myeloperoxidase (MPO) release with anti-CEACAM6 antibody F(ab)₂ fragment APP-60849 (derived from human IgG2) (black columns) and corresponding isotype control antibody fragment (white columns) with (+fMLP) and without (w/o fMLP) suboptimal fMLP stimulus. X-axis: concentration of antibody [μM]; Y-axis: pg/ml MPO.

FIG. 14: Myeloperoxidase (MPO) release with anti-CEACAM 6 antibody TPP-3310 (black columns) and corresponding isotype control antibody TPP-1238 (white columns) with (+fMLP) and without (w/o fMLP) suboptimal fMLP stimulus. Prior to addition of the anti-CEACAM6 antibody TPP-3310 or its isotype control antibody TPP-1238, a non-binding F(ab)₂ fragment matched to AT10 was added 1.4 μM concentration.

FIG. 15: Myeloperoxidase (MPO) release with anti-CEACAM 6 antibody TPP-3310 (black columns) and corresponding isotype control antibody TPP-1238 (white columns) with (+fMLP) and without (w/o fMLP) suboptimal fMLP stimulus. Prior to addition of the anti-CEACAM6 antibody TPP-3310 or its isotype control antibody TPP-1238, the blocking anti-CD32 antibody F(ab)₂ fragment AT10 was added at 1.4 μM concentration. X-axis: concentration of antibody [μM]; Y-axis: pg/ml MPO.

FIG. 16: Myeloperoxidase (MPO) release with indicated anti-CEACAM6 antibody TPP-21518 (human IgG1-LALAaglyco) (black columns) and corresponding isotype control antibodies (white columns) with (+fMLP) and without (w/o fMLP) suboptimal fMLP stimulus. X-axis: concentration of antibody [μM]; Y-axis: pg/ml MPO.

FIG. 17: Percent phagocytosis of labeled neutrophils by M2c macrophage as measured by flow cytometry after 2 hours of co-culture in presence of anti-CEACAM6 antibodies [TPP-3310 (human IgG2); TPP-21518 (human IgG1-LALAaglyco); TPP-5468 (human IgG1); TPP-1745 (9A6 human IgG1); TPP-1173 (Neo201 human IgG1)] and their corresponding isotype controls [TPP-1238 (human IgG2); TPP-21501 (human IgG1-LALAaglyco); TPP-754 (human IgG1)]. Mouse anti-huCD47 is included as positive control for phagocytosis. X-axis: Test articles with respective protein identifiers at 1 μM, 100 nM, 10 nM and 1 nM concentrations. “-”=no antibody added; CD47=mouse anti-huCD47; mlgG1=non-binding isotype control to mouse anti-huCD47; Y-axis: Percent of live, CD206-APC positive M2c macrophages that are CFSE positive from engulfment of CFSE labeled neutrophils.

### DETAILED DESCRIPTION OF THE INVENTION

### Definitions

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent with the meaning commonly understood in the art. Such references include, but are not limited to, Singleton et al., Dictionary of Microbiology and Molecular Biology (2nd ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” and “the” include plural reference unless the context clearly dictates otherwise. Thus, for example, reference to “a gene” is a reference to one or more genes and includes equivalents thereof known to those skilled in the art, and so forth.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” as used throughout the application and in particular within the claims may be replaced by the term “consisting of”.

In this context, the term “about” or “approximately” means within 80% to 120%, alternatively within 90% to 110%, including within 95% to 105% of a given value or range.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

The term “antibody”, as used herein, is intended to refer to immunoglobulin molecules. Antibodies may comprise four polypeptide chains, two heavy (H) chains (about 50-70 kDa) and two light (L) chains (about 25 kDa) which are typically inter-connected by disulfide bonds. In particular embodiments, the antibody is composed of two identical pairs of polypeptide chains.

The amino-terminal portion of each chain includes a “variable” region of about 100 to 110 or more amino acids primarily responsible for antigen recognition. The heavy chain variable region is abbreviated herein as VH, the light chain variable region is abbreviated herein as VL. The carboxyl-terminal portion of each chain defines a constant region primarily responsible for effector function. The heavy chain constant region can comprise e.g. three domains CH1, CH2 and CH3. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs, arranged from amino-terminus to carboxy-terminus e.g., in the following order. FR1, CDR1, FR2, CDR2, FR3, CDR3, FR4.

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

The term “Fc region” herein is used to define a C-terminal region of an immunoglobulin heavy chain that contains at least a portion of the constant region. An Fc region of an IgG1 comprises the CH2 and CH3 domain of an IgG1 heavy chain. The term includes native sequence Fc regions and variant Fc regions. In one embodiment, a human IgG1 heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 23-36 (L1), 52-58 (L2) and 91-101 (L3) in the light chain variable domain and 31-35 (H1), 50-65 (H2) and 98-110 (H3) in the heavy chain variable domain; (Kabat et al., Sequences of Proteins of Immunological Interest, 5th Ed. Public Health Service, National Institutes of Health, Bethesda, MD. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196:901-917 (1987)). In some instances, a complementarity determining region can include amino acids from both a CDR region defined according to Kabat and a hypervariable loop. “Framework” or FR residues are those variable domain residues other than the hypervariable region residues.

Immunoglobulins can be assigned to different classes depending on the amino acid sequence of the constant domain of their heavy chains. Heavy chains are classified as mu (μ), delta (Δ), gamma (γ), alpha (α), and epsilon (ε), and define the antibody's isotype as IgM, IgD, IgG, IgA, and IgE, respectively. In particular embodiments, the antibody according to the present invention is an IgG antibody. Several of these may be further divided into subclasses or isotypes, e.g. IgG1, IgG2, IgG3, IgG4, IgA1 and IgA2. In particular embodiments, the antibody according to the present invention is an IgG1. Different isotypes may have different effector functions. Human light chains are classified as kappa (K) and lambda (Δ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)).

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320).

Nonlimiting examples of “functional fragments” or “antigen-binding antibody fragments” include Fab, Fab′, F(ab′)₂, Fv fragments, domain antibodies (dAb), complementarity determining region (CDR) fragments, single-chain antibodies (scFv), single chain antibody fragments, diabodies, triabodies, tetrabodies, minibodies, linear antibodies (Zapata et al., Protein Eng., 8 (10): 1057-1062 (1995)); chelating recombinant antibodies, tribodies or bibodies, intrabodies, nanobodies, small modular immunopharmaceuticals (SMIPs), an antigen-binding-domain immunoglobulin fusion protein, a camelized antibody, a VHH containing antibody, or muteins or derivatives thereof, and polypeptides that contain at least a portion of an immunoglobulin that is sufficient to confer specific antigen binding to the polypeptide, such as a CDR sequence, as long as the antibody retains the desired biological activity; and multispecific antibodies such as bi- and tri-specific antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “bispecific” or “bifunctional” antibody is understood to have each of its binding sites identical. The F(ab′)₂ or Fab may be engineered to minimize or completely remove the intermolecular disulfide interactions that occur between the C_{H1} and C_{L} domains. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two “Fv” fragments. An “Fv” fragment is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen.

“Single-chain Fv” or “sFv” or “scFv” antibody fragments comprise the VH and VL domains of antibody, wherein these domains are present in a single polypeptide chain.

Preferably, the Fv polypeptide further comprises a polypeptide linker between the VH and VL domains that enables the Fv to form the desired structure for antigen binding. For a review of Fvs see Pluckthun in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteine residues from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteine residues between them.

The term “mutein” or “variant” can be used interchangeably and refers to an antibody or antigen-binding fragment that contains at least one amino acid substitution, deletion, or insertion in the variable region or the portion equivalent to the variable region, provided that the mutein or variant retains the desired binding affinity or biological activity. Variants of the antibodies or antigen-binding antibody fragments contemplated in the invention are molecules in which the binding activity of the antibody or antigen-binding antibody fragment is maintained.

A “chimeric antibody” or antigen-binding fragment thereof is defined herein as one, wherein the variable domains are derived from a non-human origin and some or all constant domains are derived from a human origin.

“Humanized antibodies” contain CDR regions derived from a non-human species, such as mouse, that have, for example, been engrafted, along with any necessary framework back-mutations, into human sequence-derived V regions. Thus, for the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. See, for example, U.S. Pat. Nos. 5,225,539; 5,585,089; 5,693,761; 5,693,762; 5,859,205, each herein incorporated by reference. In some instances, framework residues of the human immunoglobulin are replaced by corresponding non-human residues (see, for example, U.S. Pat. Nos. 5,585,089; 5,693,761; 5,693,762, each herein incorporated by reference). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-25 (1986); Riechmann et al., Nature 332:323-27 (1988); and Presta, Curr. Opin. Struct. Biol. 2:593-96 (1992), each herein incorporated by reference.

“Human antibodies” or “fully human antibodies” comprise human derived CDRs, i.e. CDRs of human origin. Fully human antibodies may comprise a low number of germline deviations compared with the closest human germline reference determined based on the IMGT database (www.imgt.org). For example, a fully human antibody according to the current invention may comprise up to 1, 2, 3, 4, 5, 6, 7, 8, 9 or 10 germline deviations in the CDRs compared with the closest human germline reference. Fully human antibodies can be developed from human derived B cells by cloning techniques in combination with a cell enrichment or immortalization step. The majority of fully human antibodies, however, are isolated either from immunized mice transgenic for the human IgG locus or from sophisticated combinatorial libraries by phage display (Brüggemann M., Osborn M. J., Ma B., Hayre J., Avis S., Lundstrom B. and Buelow R., Human Antibody Production in Transgenic Animals, Arch Immunol Ther Exp (Warsz.) 63 (2015), 101-108; Carter P. J., Potent antibody therapeutics by design, Nat Rev Immunol 6 (2006), 343-357; Frenzel A., Schirrmann T. and Hust M., Phage display-derived human antibodies in clinical development and therapy, MAbs 8 (2016), 1177-1194; Nelson A. L., Dhimolea E. and Reichert J. M., Development trends for human monoclonal antibody therapeutics, Nat Rev Drug Discov 9 (2010), 767-774.)).

Several techniques are available to generate fully human antibodies (cf. WO2008/112640 A3). Cambridge Antibody Technologies (CAT) and Dyax have obtained antibody cDNA sequences from peripheral B cells isolated from immunized humans and devised phage display libraries for the identification of human variable region sequences of a particular specificity. Briefly, the antibody variable region sequences are fused either with the Gene Ill or Gene VIII structure of the M13 bacteriophage. These antibody variable region sequences are expressed either as Fab or single chain Fv (scFv) structures at the tip of the phage carrying the respective sequences. Through rounds of a panning process using different levels of antigen binding conditions (stringencies), phages expressing Fab or scFv structures that are specific for the antigen of interest can be selected and isolated. The antibody variable region cDNA sequences of selected phages can then be elucidated using standard sequencing procedures. These sequences may then be used for the reconstruction of a full antibody having the desired isotype using established antibody engineering techniques. Antibodies constructed in accordance with this method are considered fully human antibodies (including the CDRs). In order to improve the immunoreactivity (antigen binding affinity and specificity) of the selected antibody, an in vitro maturation process can be introduced, including a combinatorial association of different heavy and light chains, deletion/addition/mutation at the CDR3 of the heavy and light chains (to mimic V-J, and V-D-J recombination), and random mutations (to mimic somatic hypermutation). An example of a “fully human” antibody generated by this method is the anti-tumor necrosis factor α antibody, Humira (adalimumab).

“Human Engineered™” antibodies generated by altering the parent sequence according to the methods set forth in Studnicka et al., U.S. Pat. No. 5,766,886.

An antibody of the invention may be derived from a recombinant antibody gene library. The development of technologies for making repertoires of recombinant human antibody genes, and the display of the encoded antibody fragments on the surface of filamentous bacteriophage, has provided a recombinant means for directly making and selecting human antibodies, which also can be applied to humanized, chimeric, murine or mutein antibodies. The antibodies produced by phage technology are produced as antigen binding fragments—usually Fv or Fab fragments—in bacteria and thus lack effector functions. Effector functions can be introduced by one of two strategies: The fragments can be engineered either into complete antibodies for expression in mammalian cells, or into bispecific antibody fragments with a second binding site capable of triggering an effector function. Typically, a heavy chain fragment (e.g. VH-CH1) and a light chain fragment (e.g. VL-CL) of antibodies are separately cloned by PCR and recombined randomly in combinatorial phage display libraries, which can then be selected for binding to a particular antigen. The Fab fragments are expressed on the phage surface, i.e., physically linked to the genes that encode them. Thus, selection of Fab by antigen binding co-selects for the Fab encoding sequences, which can be amplified subsequently. By several rounds of antigen binding and re-amplification, a procedure termed panning, Fab specific for the antigen are enriched and finally isolated.

A variety of procedures have been described for human antibodies deriving from phage display libraries. Such libraries may be built on a single master framework, into which diverse in vivo-formed (i. e. human-derived) CDRs are allowed to recombine as described by Carlsson and Söderlind Exp. Rev. Mol. Diagn. 1 (1), 102-108 (2001), Söderlin et al., Nat. Biotech. 18, 852-856 (2000) and U.S. Pat. No. 6,989,250. Alternatively, such an antibody library may be based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064. For a review of phage display screening (for example see Hoet R M et al, Nat Biotechnol 2005; 23 (3): 344-8), the well-established hybridoma technology (for example see Köhler and Milstein Nature. 1975 Aug. 7; 256 (5517): 495-7), or immunization of mice inter alia immunization of hMAb mice (e.g. Velocimmune Mouse®).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. For example, the monoclonal antibodies to be used may be made by the hybridoma method first described by Kohler et al., Nature, 256:495 [1975, or may be made by recombinant DNA methods (see, e.g., U.S. Pat. No. 4,816,567). The “monoclonal antibodies” may also be recombinant, chimeric, humanized, human, Human Engineered™, or antibody fragments, for example.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or non-proteinaceous solutes.

An “isolated” nucleic acid is one that has been identified and separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “anti-antigen” antibody refers to an antibody that specifically binds to the antigen. For example, an anti-PD-1 antibody specifically binds to PD-1 and an anti-CECAM6 antibody specifically binds to CECAM6.

As used herein, an antibody “specifically binds to”, is “specific to/for” or “specifically recognizes” an antigen of interest, e.g. CEACAM6, is one that binds the antigen with sufficient affinity such that the antibody is useful as a therapeutic agent in targeting a cell or tissue expressing the antigen, and does not significantly cross-react with proteins other than orthologs and variants (e.g. mutant forms, splice variants, or proteolytically truncated forms) of the aforementioned antigen target. The term “specifically recognizes” or “specifically binds to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁷ M, alternatively less than about 10⁻¹⁰ M, alternatively less than about 10⁻¹¹ M, alternatively less than about 10⁻² M, or less. An antibody “specifically binds to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using a Biacore T200 instrument (GE Healthcare Biacore, Inc.). Other suitable devices are BIACORE T100, BIACORE (R)-2000, BIACORe 4000, a BIACORE (R)-3000 (BIAcore, Inc., Piscataway, NJ), or ProteOn XPR36 instrument (Bio-Rad Laboratories, Inc.).

As used herein, the term “epitope” includes any protein determinant capable of specific binding to an antibody, an immunoglobulin or T-cell receptor. Epitopic determinants usually consist of chemically active surface groupings of molecules such as amino acids or sugar side chains, or combinations thereof and usually have specific three dimensional structural characteristics, as well as specific charge characteristics.

The term “antibody that binds to the same epitope” as a reference antibody or “an antibody which competes for binding” or the term “compete” when used in the context of antigen binding proteins (e.g., antibodies) that compete for the same epitope means competition between antigen binding proteins as determined by an assay in which the antigen binding protein (e.g., antibody or immunologically functional fragment thereof) being tested prevents or inhibits (e.g., reduces) specific binding of a reference antigen binding protein (e.g., a ligand, or a reference antibody) to a common antigen (e.g., CEACAM6 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “maturated antibodies” or “maturated antigen-binding fragments” such as maturated Fab variants or “optimized” variants includes derivatives of an antibody or antibody fragment exhibiting stronger binding—i. e. binding with increased affinity—to a given antigen such as the extracellular domain of a target protein. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

“Sequence homology” indicates the percentage of amino acids that either is identical or that represent conservative amino acid substitutions.

An “antagonistic” antibody or a “blocking” antibody is one which significantly inhibits (either partially or completely) a biological activity of the antigen it binds. In particular embodiments, the antibody or antigen-binding fragment according to the present invention is a CEACAM6 blocking antibody or antigen-binding fragment.

The term “antibody conjugate” refers to an antibody conjugated to one or more molecules including drugs—in which case the antibody conjugate is referred to as “antibody-drug conjugate” (“ADC”)—and high molecular weight molecules such as peptides or proteins.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell”, “host cell line”, and “host cell culture” are used interchangeably and refer to cells into which at least one exogenous nucleic acid has been introduced, including the progeny of such cells. Host cells include “transformants” and “transformed cells”, “transfectants” and “transfected cells” and “transduced cells” which include the primary transformed/transfected/transduced cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

As used herein, the phrase “therapeutically effective amount” is meant to refer to an amount of therapeutic or prophylactic antibody that would be appropriate to elicit the desired therapeutic or prophylactic effect or response, including alleviating some or all of such symptoms of disease or reducing the predisposition to the disease, when administered in accordance with the desired treatment regimen.

The term “pharmaceutical formulation”/“pharmaceutical composition” refers to a preparation which is in such form as to permit the biological activity of an active ingredient contained therein to be effective, and which contains no additional components which are unacceptably toxic to a subject to which the formulation would be administered.

As used herein “CEACAM6” designates the “carcinoembryonic antigen-related cell adhesion molecule 6”, also known as “CD66c” (Cluster of Differentiation 66c), or Non-specific crossreacting antigen, or NCA, or NCA-50/90. CEACAM6 is a glycosylphosphatidylinositol (GPI)-linked cell surface protein involved in cell-cell adhesion. The term “CEACAM6” as used herein includes human CEACAM6 (hCEACAM6), variants, isoforms, and species homologs (orthologs) of hCEACAM6. A reference sequence for human CEACAM6 (hCEACAM6) is available from UniProtKB/Swiss-Prot data base under accession number P40199.3 and from NCBI under Reference Sequence: NP_002474.4. The mature extracellular region of human CEACAM6 consists of amino acids at position 35-320 of SEQ-ID No: 75. Domain 1 of human CEACAM6 (also known as N domain, also known as N-terminal domain 1) consists of amino acids at position 35-142 of SEQ-ID No: 75.

The terms “anti-CEACAM6 antibody” and “an antibody that binds to CEACAM6” refer to an antibody that is capable of binding human CEACAM6 with sufficient affinity such that the antibody is useful as a diagnostic and/or therapeutic agent in targeting CEACAM6. In one embodiment, the extent of binding of an anti CEACAM6 antibody to an unrelated, non-CEACAM6 protein is less than about 10%, less than about 5%, or less than about 2% of the binding of the antibody to CEACAM6 as measured, e.g., by standard ELISA procedure. In certain embodiments, an antibody that binds to CEACAM6 has a binding activity (EC50) of ≤1 uM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-CEACAM6 antibody binds to an epitope of CEACAM6 that is conserved among CEACAM6 from different species.

“Programmed Death-1 (PD-1)” refers to an immunoinhibitory receptor belonging to the CD28 family. PD-1 is expressed predominantly on previously activated T cells in vivo and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that down regulate T cell activation and cytokine secretion upon binding to PD-1. The term “PD-L1” as used herein includes human PD-L1 (hPDL1), variants, isoforms, and species homologs of hPD-L1, and analogs having at least one common epitope with hPD-L1. The complete hPD-L1 sequence can be found under GenBank Accession No. Q9NZQ7.

As used herein “TIM-3” designates the “T cell immunoglobulin domain and mucin domain 3” (also known as HAVCAR2) a member of the TIM-family. TIM-3 is a transmembrane protein on the cell surface. It has been described as an activation-induced inhibitory molecule involved in tolerance and shown to induce T cell exhaustion. The term “TIM-3” as used herein includes human TIM-3 (hTIM-3), variants, isoforms, and species homologs of hTIM-3, and analogs having at least one common eptope with hTIM-3. A reference sequence for human TIM-3 is available from UniProtKB/Swiss-Prot data base under accession number UniProtKB QBTDOO (HAVR2_HUMAN) and NCBI database, NCBI Reference Sequence: NP_116171.3.

TABLE 0
Brief description of sequences:

| TPP ID | Region | SEQ ID | Sequence |
|---|---|---|---|
| TPP-1173 | VH | SEQ ID NO: 1 | QVQLVQSGAEVKKPGASVKVSCKASGYTFTDYAM |
| | | | HWVRQAPGQRLEWMGLISTYSGDTKYNQNFQGRV |
| | | | TMTVDKSASTAYMELSSLRSEDTAVYYCARGDYS |
| | | | GSRYWFAYWGQGTLVTVSS |
| TPP-1173 | HCDR1 | SEQ ID NO: 2 | DYAMH |
| TPP-1173 | HCDR2 | SEQ ID NO: 3 | LISTYSGDTKYNQNFQG |
| TPP-1173 | HCDR3 | SEQ ID NO: 4 | GDYSGSRYWFAY |
| TPP-1173 | VL | SEQ ID NO: 5 | DIQMTQSPSSLSASVGDRVTITCQASENIYGALN |
| | | | WYQRKPGKSPKLLIYGASNLATGMPSRFSGSGSG |
| | | | TDYTFTISSLQPEDIATYYCQQVLSSPYTFGGGT |
| | | | KLEIK |
| TPP-1173 | LCDR1 | SEQ ID NO: 6 | QASENIYGALN |
| TPP-1173 | LCDR2 | SEQ ID NO: 7 | GASNLAT |
| TPP-1173 | LCDR3 | SEQ ID NO: 8 | QQVLSSPYT |
| TPP-1173 | Heavy Chain | SEQ ID NO: 9 | QVQLVQSGAEVKKPGASVKVSCKASGYTFTDYAM |
| | | | HWVRQAPGQRLEWMGLISTYSGDTKYNQNFQGRV |
| | | | TMTVDKSASTAYMELSSLRSEDTAVYYCARGDYS |
| | | | GSRYWFAYWGQGTLVTVSSASTKGPSVFPLAPSS |
| | | | KSTSGGTAALGCLVKDYFPEPVTVSWNSGALTSG |
| | | | VHTFPAVLQSSGLYSLSSVVTVPSSSLGTQTYIC |
| | | | NVNHKPSNTKVDKKVEPKSCDKTHTCPPCPAPEL |
| | | | LGGPSVFLFPPKPKDTLMISRTPEVTCVVVDVSH |
| | | | EDPEVKFNWYVDGVEVHNAKTKPREEQYNSTYRV |
| | | | VSVLTVLHQDWLNGKEYKCKVSNKALPAPIEKTI |
| | | | SKAKGQPREPQVYTLPPSRDELTKNQVSLTCLVK |
| | | | GFYPSDIAVEWESNGQPENNYKTTPPVLDSDGSF |
| | | | FLYSKLTVDKSRWQQGNVFSCSVMHEALHNHYTQ |
| | | | KSLSLSPG |
| TPP-1173 | Light Chain | SEQ ID NO: 10 | DIQMTQSPSSLSASVGDRVTITCQASENIYGALN |
| | | | WYQRKPGKSPKLLIYGASNLATGMPSRFSGSGSG |
| | | | TDYTFTISSLQPEDIATYYCQQVLSSPYTFGGGT |
| | | | KLEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP-3310 | VH | SEQ ID NO: 11 | QVTLRESGPALVKPTQTLTLTCTFSGFSLSTYGI |
| | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSS |
| TPP-3310 | HCDR1 | SEQ ID NO: 12 | TYGIGVG |
| TPP-3310 | HCDR2 | SEQ ID NO: 13 | HIWWNDNKYYSTSLKT |
| TPP-3310 | HCDR3 | SEQ ID NO: 14 | ISLPYFDY |
| TPP-3310 | VL | SEQ ID NO: 15 | DIQLTQSPSFLSASVGDRVTITCKASQNVGTAVA |
| | | | WYQQKPGKAPKLLIYSASNRYTGVPSRFSGSGSG |
| | | | TEFTLTISSLQPEDFATYYCQQYSSYPLTFGGGT |
| | | | KVEIK |
| TPP-3310 | LCDR1 | SEQ ID NO: 16 | KASQNVGTAVA |
| TPP-3310 | LCDR2 | SEQ ID NO: 17 | SASNRYT |
| TPP-3310 | LCDR3 | SEQ ID NO: 18 | QQYSSYPLT |
| TPP-3310 | Heavy Chain | SEQ ID NO: 19 | QVTLRESGPALVKPTQTLTLTCTFSGFSLSTYGI |
| | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSSASTKGPSVFPLAPCSRST |
| | | | SESTAALGCLVKDYFPEPVTVSWNSGALTSGVHT |
| | | | FPAVLQSSGLYSLSSVVTVPSSNFGTQTYTCNVD |
| | | | HKPSNTKVDKTVERKCCVECPPCPAPPVAGPSVF |
| | | | LFPPKPKDTLMISRTPEVTCVVVDVSHEDPEVQE |
| | | | NWYVDGVEVHNAKTKPREEQFNSTFRVVSVLTVV |
| | | | HQDWLNGKEYKCKVSNKGLPAPIEKTISKTKGQP |
| | | | REPQVYTLPPSREEMTKNQVSLTCLVKGFYPSDI |
| | | | AVEWESNGQPENNYKTTPPMLDSDGSFFLYSKLT |
| | | | VDKSRWQQGNVFSCSVMHEALHNHYTQKSLSLSP |
| | | | G |
| TPP-3310 | Light Chain | SEQ ID NO: 20 | DIQLTQSPSFLSASVGDRVTITCKASQNVGTAVA |
| | | | WYQQKPGKAPKLLIYSASNRYTGVPSRFSGSGSG |
| | | | TEFTLTISSLQPEDFATYYCQQYSSYPLTFGGGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP-3470 | Heavy Chain | SEQ ID NO: 21 | QVTLKESGPGILQPSQTLTLTCSFSGFSLSTSGL |
| | | | SVGWIRQPSGKGLEWLAHIWWNDDKYYNPALKSR |
| | | | LTISKVTSNNQVFLKIASVVTADTATYYCARIPR |
| | | | GAMDYWGQGTSVTVSSASTKGPSVFPLAPCSRST |
| | | | SESTAALGCLVKDYFPEPVTVSWNSGALTSGVHT |
| | | | FPAVLQSSGLYSLSSVVTVPSSNFGTQTYTCNVD |
| | | | HKPSNTKVDKTVERKCCVECPPCPAPPVAGPSVF |
| | | | LFPPKPKDTLMISRTPEVTCVVVDVSHEDPEVQF |
| | | | NWYVDGVEVHNAKTKPREEQFNSTERVVSVLTVV |
| | | | HQDWLNGKEYKCKVSNKGLPAPIEKTISKTKGQP |
| | | | REPQVYTLPPSREEMTKNQVSLTCLVKGFYPSDI |
| | | | AVEWESNGQPENNYKTTPPMLDSDGSFFLYSKLT |
| | | | VDKSRWQQGNVFSCSVMHEALHNHYTQKSLSLSP |
| | | | G |
| TPP-3470 | Light Chain | SEQ ID NO: 22 | DIVMTQSQKEMSTSVGDRVSVTCKASQNVYTNVA |
| | | | WYQQKPGQSPKALIYSASYRYSGVPDRFTGSGSG |
| | | | TDFTLTITNVQSEDLAEYFCQQYNSYPLTFGSGT |
| | | | KLEIKRADAAPTVSIFPPSSEQLTSGGASVVCFL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP-3688 | VH | SEQ ID NO: 23 | QVQLVQSGAEVKKPGASVKVSCKASGYTFTDYAM |
| | | | HWVRQAPGQRLEWMGLISTYSGDTKYNQNFQGRV |
| | | | TMTVDKSASTAYMELSSLRSEDTAVYYCARGDYS |
| | | | GSRYWFAYWGQGTLVTVSS |
| TPP-3688 | HCDR1 | SEQ ID NO: 24 | DYAMH |
| TPP-3688 | HCDR2 | SEQ ID NO: 25 | LISTYSGDTKYNQNFQG |
| TPP-3688 | HCDR3 | SEQ ID NO: 26 | GDYSGSRYWFAY |
| TPP-3688 | VL | SEQ ID NO: 27 | DIQMTQSPSSLSASVGDRVTITCQASENIYGALN |
| | | | WYQRKPGKSPKLLIYGASNLATGMPSRFSGSGSG |
| | | | TDYTFTISSLQPEDIATYYCQQVLSSPYTFGGGT |
| | | | KLEIK |
| TPP-3688 | LCDR1 | SEQ ID NO: 28 | QASENIYGALN |
| TPP-3688 | LCDR2 | SEQ ID NO: 29 | GASNLAT |
| TPP-3688 | LCDR3 | SEQ ID NO: 30 | QQVLSSPYT |
| TPP-3688 | Heavy Chain | SEQ ID NO: 31 | QVQLVQSGAEVKKPGASVKVSCKASGYTFTDYAM |
| | | | HWVRQAPGQRLEWMGLISTYSGDTKYNQNFQGRV |
| | | | TMTVDKSASTAYMELSSLRSEDTAVYYCARGDYS |
| | | | GSRYWFAYWGQGTLVTVSSASTKGPSVFPLAPCS |
| | | | RSTSESTAALGCLVKDYFPEPVTVSWNSGALTSG |
| | | | VHTFPAVLQSSGLYSLSSVVTVPSSNFGTQTYTC |
| | | | NVDHKPSNTKVDKTVERKCCVECPPCPAPPVAGP |
| | | | SVFLFPPKPKDTLMISRTPEVTCVVVDVSHEDPE |
| | | | VQFNWYVDGVEVHNAKTKPREEQENSTFRVVSVL |
| | | | TVVHQDWLNGKEYKCKVSNKGLPAPIEKTISKTK |
| | | | GQPREPQVYTLPPSREEMTKNQVSLTCLVKGFYP |
| | | | SDIAVEWESNGQPENNYKTTPPMLDSDGSFFLYS |
| | | | KLTVDKSRWQQGNVFSCSVMHEALHNHYTQKSLS |
| | | | LSPG |
| TPP-3688 | Light Chain | SEQ ID NO: 32 | DIQMTQSPSSLSASVGDRVTITCQASENIYGALN |
| | | | WYQRKPGKSPKLLIYGASNLATGMPSRFSGSGSG |
| | | | TDYTFTISSLQPEDIATYYCQQVLSSPYTFGGGT |
| | | | KLEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP-5468 | VH | SEQ ID NO: 33 | QVTLRESGPALVKPTQTLTLTCTFSGFSLSTYGI |
| | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSS |
| TPP-5468 | HCDR1 | SEQ ID NO: 34 | TYGIGVG |
| TPP-5468 | HCDR2 | SEQ ID NO: 35 | HIWWNDNKYYSTSLKT |
| TPP-5468 | HCDR3 | SEQ ID NO: 36 | ARISLPYFDY |
| TPP-5468 | VL | SEQ ID NO: 37 | DIQLTQSPSFLSASVGDRVTITCKASQNVGTAVA |
| | | | WYQQKPGKAPKLLIYSASNRYTGVPSRFSGSGSG |
| | | | TEFTLTISSLQPEDFATYYCQQYSSYPLTFGGGT |
| | | | KVEIK |
| TPP-5468 | LCDR1 | SEQ ID NO: 38 | KASQNVGTAVA |
| TPP-5468 | LCDR2 | SEQ ID NO: 39 | SASNRYT |
| TPP-5468 | LCDR3 | SEQ ID NO: 40 | QQYSSYPLT |
| TPP-5468 | Heavy Chain | SEQ ID NO: 41 | QVTLRESGPALVKPTQTLTLTCTFSGFSLSTYGI |
| | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSSASTKGPSVFPLAPSSKST |
| | | | SGGTAALGCLVKDYFPEPVTVSWNSGALTSGVHT |
| | | | FPAVLQSSGLYSLSSVVTVPSSSLGTQTYICNVN |
| | | | HKPSNTKVDKKVEPKSCDKTHTCPPCPAPELLGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPG |
| TPP-5468 | Light Chain | SEQ ID NO: 42 | DIQLTQSPSFLSASVGDRVTITCKASQNVGTAVA |
| | | | WYQQKPGKAPKLLIYSASNRYTGVPSRFSGSGSG |
| | | | TEFTLTISSLQPEDFATYYCQQYSSYPLTFGGGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP- | VH | SEQ ID NO: 43 | QVTLRESGPALVKPTQTLTLTCTFSGFSLSTYGI |
| 10914 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSS |
| TPP- | HCDR1 | SEQ ID NO: 44 | TYGIGVG |
| 10914 | | | |
| TPP- | HCDR2 | SEQ ID NO: 45 | HIWWNDNKYYSTSLKT |
| 10914 | | | |
| TPP- | HCDR3 | SEQ ID NO: 46 | ISLPYFDY |
| 10914 | | | |
| TPP- | VL | SEQ ID NO: 47 | DIQLTQSPSFLSASVGDRVTITCKASQNVGTAVA |
| 10914 | | | WYQQKPGKAPKLLIYSASNRYTGVPSRFSGSGSG |
| | | | TEFTLTISSLQPEDFATYYCQQYSSYPLTFGGGT |
| | | | KVEIK |
| TPP- | LCDR1 | SEQ ID NO: 48 | KASQNVGTAVA |
| 10914 | | | |
| TPP- | LCDR2 | SEQ ID NO: 49 | SASNRYT |
| 10914 | | | |
| TPP- | LCDR3 | SEQ ID NO: 50 | QQYSSYPLT |
| 10914 | | | |
| TPP- | Heavy Chain | SEQ ID NO: 51 | QVTLRESGPALVKPTQTLTLTCTFSGFSLSTYGI |
| 10914 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSSASTKGPSVFPLAPSSKST |
| | | | SGGTAALGCLVKDYFPEPVTVSWNSGALTSGVHT |
| | | | FPAVLQSSGLYSLSSVVTVPSSSLGTQTYICNVN |
| | | | HKPSNTKVDKKVEPKSCDKTHTCPPCPAPELLGG |
| | | | PSVFLFPPKPKDTLMISRTPEVTCVVVDVSHEDP |
| | | | EVKFNWYVDGVEVHNAKTKPREEQYASTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPG |
| TPP- | Light Chain | SEQ ID NO: 52 | DIQLTQSPSFLSASVGDRVTITCKASQNVGTAVA |
| 10914 | | | WYQQKPGKAPKLLIYSASNRYTGVPSRFSGSGSG |
| | | | TEFTLTISSLQPEDFATYYCQQYSSYPLTFGGGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP- | VH | SEQ ID NO: 53 | QVTLRESGPALVKPTQTLTLTCTFSGFSLSTYGI |
| 19919 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSS |
| TPP- | HCDR1 | SEQ ID NO: 54 | TYGIGVG |
| 19919 | | | |
| TPP- | HCDR2 | SEQ ID NO: 55 | HIWWNDNKYYSTSLKT |
| 19919 | | | |
| TPP- | HCDR3 | SEQ ID NO: 56 | ISLPYFDY |
| 19919 | | | |
| TPP- | VL | SEQ ID NO: 57 | DIQLTQSPSFLSASVGDRVTITCKASQNVGTAVA |
| 19919 | | | WYQQKPGKAPKLLIYSASNRYTGVPSRFSGSGSG |
| | | | TEFTLTISSLQPEDFATYYCQQYSSYPLTFGGGT |
| | | | KVEIK |
| TPP- | LCDR1 | SEQ ID NO: 58 | KASQNVGTAVA |
| 19919 | | | |
| TPP- | LCDR2 | SEQ ID NO: 59 | SASNRYT |
| 19919 | | | |
| TPP- | LCDR3 | SEQ ID NO: 60 | QQYSSYPLT |
| 19919 | | | |
| TPP- | Heavy Chain | SEQ ID NO: 61 | QVTLRESGPALVKPTQTLTLTCTFSGFSLSTYGI |
| 19919 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSSASTKGPSVFPLAPSSKST |
| | | | SGGTAALGCLVKDYFPEPVTVSWNSGALTSGVHT |
| | | | FPAVLQSSGLYSLSSVVTVPSSSLGTQTYICNVN |
| | | | HKPSNTKVDKKVEPKSCDKTHTCPPCPAPEAAGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPGK |
| TPP- | Light Chain | SEQ ID NO: 62 | DIQLTQSPSFLSASVGDRVTITCKASQNVGTAVA |
| 19919 | | | WYQQKPGKAPKLLIYSASNRYTGVPSRFSGSGSG |
| | | | TEFTLTISSLQPEDFATYYCQQYSSYPLTFGGGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP- | VH | SEQ ID NO: 63 | QVTLRESGPALVKPTQTLTLTCTFSGFSLSTYGI |
| 21518 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSS |
| TPP- | HCDR1 | SEQ ID NO: 64 | TYGIGVG |
| 21518 | | | |
| TPP- | HCDR2 | SEQ ID NO: 65 | HIWWNDNKYYSTSLKT |
| 21518 | | | |
| TPP- | HCDR3 | SEQ ID NO: 66 | ISLPYFDY |
| 21518 | | | |
| TPP- | VL | SEQ ID NO: 67 | DIQLTQSPSFLSASVGDRVTITCKASQNVGTAVA |
| 21518 | | | WYQQKPGKAPKLLIYSASNRYTGVPSRFSGSGSG |
| | | | TEFTLTISSLQPEDFATYYCQQYSSYPLTFGGGT |
| | | | KVEIK |
| TPP- | LCDR1 | SEQ ID NO: 68 | KASQNVGTAVA |
| 21518 | | | |
| TPP- | LCDR2 | SEQ ID NO: 69 | SASNRYT |
| 21518 | | | |
| TPP- | LCDR3 | SEQ ID NO: 70 | QQYSSYPLT |
| 21518 | | | |
| TPP- | Heavy Chain | SEQ ID NO: 71 | QVTLRESGPALVKPTQTLTLTCTFSGFSLSTYGI |
| 21518 | | | GVGWIRQPPGKALEWLAHIWWNDNKYYSTSLKTR |
| | | | LTISKDTSKNQVVLTMTNMDPVDTATYYCARISL |
| | | | PYFDYWGQGTTLTVSSASTKGPSVFPLAPSSKST |
| | | | SGGTAALGCLVKDYFPEPVTVSWNSGALTSGVHT |
| | | | FPAVLQSSGLYSLSSVVTVPSSSLGTQTYICNVN |
| | | | HKPSNTKVDKKVEPKSCDKTHTCPPCPAPEAAGG |
| | | | PSVFLFPPKPKDTLMISRTPEVTCVVVDVSHEDP |
| | | | EVKFNWYVDGVEVHNAKTKPREEQYASTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPGK |
| TPP- | Light Chain | SEQ ID NO: 72 | DIQLTQSPSFLSASVGDRVTITCKASQNVGTAVA |
| 21518 | | | WYQQKPGKAPKLLIYSASNRYTGVPSRFSGSGSG |
| | | | TEFTLTISSLQPEDFATYYCQQYSSYPLTFGGGT |
| | | | KVEIKRTVAAPSVFIFPPSDEQLKSGTASVVCLL |
| | | | NNFYPREAKVQWKVDNALQSGNSQESVTEQDSKD |
| | | | STYSLSSTLTLSKADYEKHKVYACEVTHQGLSSP |
| | | | VTKSFNRGEC |
| TPP- | Heavy Chain | SEQ ID NO: 73 | CAGGTCACACTGAGAGAGTCCGGCCCTGCCCTGG |
| 21518 | | (DNA) | TGAAACCCACCCAGACCCTGACCCTGACATGCAC |
| | | | CTTCAGCGGCTTCAGCCTGAGCACCTACGGCATC |
| | | | GGCGTGGGCTGGATCAGACAGCCCCCTGGCAAGG |
| | | | CCCTGGAATGGCTGGCCCACATCTGGTGGAACGA |
| | | | CAACAAGTACTACAGCACCAGCCTGAAAACCCGG |
| | | | CTGACCATCAGCAAGGACACCAGCAAGAACCAGG |
| | | | TGGTGCTGACCATGACCAACATGGACCCCGTGGA |
| | | | CACCGCCACCTACTACTGCGCCCGGATCAGCCTG |
| | | | CCCTACTTCGACTACTGGGGCCAGGGCACCACCC |
| | | | TGACCGTGTCCTCAGCCAGCACAAAGGGCCCTAG |
| | | | CGTGTTCCCTCTGGCTCCTAGCAGCAAGTCTACA |
| | | | AGCGGAGGAACAGCCGCTCTGGGCTGCCTGGTCA |
| | | | AGGATTACTTTCCCGAGCCTGTGACCGTGTCCTG |
| | | | GAATTCTGGCGCTCTGACAAGCGGCGTGCACACC |
| | | | TTTCCAGCTGTGCTGCAAAGCAGCGGCCTGTACT |
| | | | CTCTGAGCAGCGTGGTCACAGTGCCAAGCTCTAG |
| | | | CCTGGGCACCCAGACCTACATCTGCAATGTGAAC |
| | | | CACAAGCCTAGCAACACCAAGGTGGACAAGAAGG |
| | | | TGGAACCCAAGAGCTGCGACAAGACCCACACCTG |
| | | | TCCTCCATGTCCTGCTCCAGAAGCTGCTGGCGGC |
| | | | CCTTCCGTGTTTCTGTTCCCTCCAAAGCCTAAGG |
| | | | ACACCCTGATGATCAGCAGAACCCCTGAAGTGAC |
| | | | CTGCGTGGTGGTGGATGTGTCCCACGAGGATCCC |
| | | | GAAGTGAAGTTCAATTGGTACGTGGACGGCGTGG |
| | | | AAGTGCACAACGCCAAGACCAAGCCTAGAGAGGA |
| | | | ACAGTACGCCAGCACCTACAGAGTGGTGTCCGTG |
| | | | CTGACAGTGCTGCACCAGGATTGGCTGAACGGCA |
| | | | AAGAGTACAAGTGCAAGGTGTCCAACAAGGCCCT |
| | | | GCCTGCTCCTATCGAGAAAACCATCAGCAAGGCC |
| | | | AAGGGCCAGCCTAGGGAACCCCAGGTTTACACAC |
| | | | TGCCTCCAAGCAGGGACGAGCTGACCAAGAATCA |
| | | | GGTGTCCCTGACCTGCCTCGTGAAGGGCTTCTAC |
| | | | CCTTCCGATATCGCCGTGGAATGGGAGAGCAATG |
| | | | GCCAGCCTGAGAACAACTACAAGACAACCCCTCC |
| | | | TGTGCTGGACAGCGACGGCTCATTCTTCCTGTAC |
| | | | AGCAAGCTGACCGTGGACAAGTCCAGATGGCAGC |
| | | | AGGGCAACGTGTTCAGCTGCAGCGTGATGCACGA |
| | | | GGCCCTGCACAACCACTACACCCAGAAGTCCCTG |
| | | | AGCCTGTCTCCTGGCAAG |
| TPP- | Light Chain | SEQ ID NO: 74 | GACATCCAGCTGACCCAGAGCCCCAGCTTCCTGA |
| 21518 | | (DNA) | GCGCCAGCGTGGGCGACAGAGTGACCATCACATG |
| | | | CAAGGCCAGCCAGAACGTGGGCACCGCCGTGGCC |
| | | | TGGTATCAGCAGAAGCCCGGCAAGGCCCCCAAGC |
| | | | TGCTGATCTACAGCGCCAGCAACCGGTACACCGG |
| | | | CGTGCCCAGCAGATTCAGCGGCAGCGGCTCCGGC |
| | | | ACCGAGTTCACCCTGACCATCAGCAGCCTGCAGC |
| | | | CCGAGGACTTCGCCACCTACTACTGCCAGCAGTA |
| | | | CAGCAGCTACCCCCTGACCTTCGGCGGAGGCACC |
| | | | AAGGTGGAAATCAAACGAACCGTGGCCGCTCCCA |
| | | | GCGTGTTCATCTTCCCACCTAGCGACGAGCAGCT |
| | | | GAAGTCCGGCACAGCCTCTGTCGTGTGCCTGCTG |
| | | | AACAACTTCTACCCCCGCGAGGCCAAGGTGCAGT |
| | | | GGAAGGTGGACAATGCCCTGCAGAGCGGCAACAG |
| | | | CCAGGAAAGCGTGACCGAGCAGGACAGCAAGGAC |
| | | | TCCACCTACAGCCTGAGCAGCACCCTGACCCTGA |
| | | | GCAAGGCCGACTACGAGAAGCACAAGGTGTACGC |
| | | | CTGCGAAGTGACCCACCAGGGCCTGTCTAGCCCC |
| | | | GTGACCAAGAGCTTCAACCGGGGCGAGTGT |
| hCEACAM6 | | SEQ ID NO: 75 | MGPPSAPPCRLHVPWKEVLLTASLLTFWNPPTTA |
| | | | KLTIESTPFNVAEGKEVLLLAHNLPQNRIGYSWY |
| | | | KGERVDGNSLIVGYVIGTQQATPGPAYSGRETIY |
| | | | PNASLLIQNVTQNDTGFYTLQVIKSDLVNEEATG |
| | | | QFHVYPELPKPSISSNNSNPVEDKDAVAFTCEPE |
| | | | VQNTTYLWWVNGQSLPVSPRLQLSNGNMTLTLLS |
| | | | VKRNDAGSYECEIQNPASANRSDPVTLNVLYGPD |
| | | | GPTISPSKANYRPGENLNLSCHAASNPPAQYSWF |
| | | | INGTFQQSTQELFIPNITVNNSGSYMCQAHNSAT |
| | | | GLNRTTVTMITVSGSAPVLSAVATVGITIGVLAR |
| | | | VALI |

### First Aspect of the Invention: Anti-CECAM6 Antibody

The present invention relates to antibodies, that bind to human CEACAM6 (anti-CECAM6 antibodies) and are able to relieve CEACAM6-mediated immunosuppression, wherein said antibodies have reduced side-effects during treatment.

Of particular interest in the present invention are the Fc regions of said anti-CEACAM6 antibodies. By “Fc” or “Fc region”, as used herein is meant the polypeptide comprising the constant region of an antibody heavy chain excluding the first constant region immunoglobulin domain CH1 and in some cases, part of the hinge. Thus Fc refers to the last two constant region immunoglobulin domains CH2 and CH3. Although the boundaries of the Fc region may vary, the human IgG heavy chain Fc region is usually defined to include residues C226 or P230 to its carboxyl-terminus, wherein the numbering is according to the EU index as in Kabat. An IgG1 Fc region is a Fc region from an antibody of the IgG1 isotype.

In a first aspect, the present invention relates to an anti-CECAM6 antibody comprising an IgG1 Fc region lacking the glycans attached to the conserved N-linked site in the CH2 domains of the Fc region, wherein said IgG1 Fc region comprises at least the amino acid substitutions L234A and L235A, as numbered according to the EU index of Kabat. Antibodies lacking the glycans attached to the conserved N-linked site in the CH2 domains are also called aglycosyl antibodies or aglyco antibodies. The conserved N-linked glycosylation occurs at N297, as numbered according to the EU index of Kabat

In one embodiment of present invention, the modification comprises a mutation at the heavy chain glycosylation site to prevent glycosylation at the site. Thus, in one preferred embodiment of this invention, the aglycosyl antibodies or antibody derivatives are prepared by mutation of the heavy chain glycosylation site, —i.e., mutation of N297 using Kabat EU numbering and expressed in an appropriate host cell.

In certain embodiments of the first aspect the invention provides an anti-CECAM6 antibody comprising an IgG1 Fc region, wherein said IgG1 Fc region comprises an amino acid substitution N297A, N297G, or N297Q as numbered according to the EU index of Kabat. An anti-CECAM6 antibody comprising an IgG1 Fc region, wherein said IgG1 Fc region comprises an amino acid substitution N297A, N297G, or N297Q as numbered according to the EU index of Kabat, is an antibody lacking the glycans attached to the conserved N-linked site in the CH2 domain, without further mentioning that the glycans are lacking.

In another embodiment of the present invention, aglycosyl antibodies are generated by methods comprising expressing the antibodies in host cells which are incapable of attaching glycans to Asn residues, e.g. by using procaryotic host cells or by using eucaryotic host cells which are modified to lack the necessary enzymes.

In another embodiment of the present invention, aglycosyl antibodies are generated by methods comprising expressing the antibodies in in vitro methods which do not have the N-glycosylation capabilities.

In another embodiment of the present invention, aglycosyl antibodies are generated by methods comprising the removal of the CH2 domain linked glycans, —i.e., deglycosylation. These aglycosyl antibodies may be generated by conventional methods and then deglycosylated enzymatically. Methods for enzymatic deglycosylation of antibodies are well known in the art (e.g. Winkelhake & Nicolson (1976), J Biol Chem. 251 (4): 1074-80).

In another embodiment of this invention, deglycosylation may be achieved using the glycosylation inhibitor tunicamycin (Nose & Wigzell (1983), Proc Natl Acad Sci USA, 80 (21): 6632-6). That is, the modification is the prevention of glycosylation at the conserved N-linked site in the CH2 domains of the Fc portion of said antibody.

In certain embodiments of the first aspect the invention provides an anti-CECAM6 antibody comprising an IgG1 Fc region, wherein said IgG1 Fc region comprises an amino acid substitution N297A, N297G, or N297Q and at least the amino acid substitutions L234A and L235A, as numbered according to the EU index of Kabat.

In certain embodiments of the first aspect the invention provides an anti-CECAM6 antibody comprising an IgG1 Fc region, wherein said IgG1 Fc region comprises at least the amino acid substitutions N297A, L234A, and L235A, as numbered according to the EU index of Kabat.

In certain embodiments of the first aspect the invention provides an anti-CECAM6 antibody comprising an IgG1 Fc region, wherein said IgG1 Fc region comprises the amino acid substitutions N297A, L234A, and L235A, as numbered according to the EU index of Kabat.

In certain embodiments of the first aspect the anti-CECAM6 antibody mentioned supra competes for CEACAM6 binding with an antibody comprising a heavy chain variable region (VH) comprising the amino acid sequence of Seq ID No: 63 and a light chain variable region (VL) comprising the amino acid sequence of Seq ID No: 67.

In certain embodiments of the first aspect the anti-CECAM6 antibody mentioned supra comprises: a heavy chain variable region H-CDR1 comprising the amino acid sequence of SEQ ID NO: 64, a heavy chain variable region H-CDR2 comprising the amino acid sequence of SEQ ID NO: 65, a heavy chain variable region H-CDR3 comprising the amino acid sequence of SEQ ID NO: 66, a light chain variable region L-CDR1 comprising the amino acid sequence of SEQ ID NO: 68, a light chain variable region L-CDR2 comprising the amino acid sequence of SEQ ID NO: 69, and a light chain variable region L-CDR3 comprising the amino acid sequence of SEQ ID NO: 70.

In certain embodiments of the first aspect the anti-CECAM6 antibody mentioned supra comprises: a heavy chain variable region H-CDR1 amino acid sequence of SEQ ID NO: 64, a heavy chain variable region H-CDR2 amino acid sequence of SEQ ID NO: 65, a heavy chain variable region H-CDR3 amino acid sequence of SEQ ID NO: 66, a light chain variable region L-CDR1 amino acid sequence of SEQ ID NO: 68, a light chain variable region L-CDR2 amino acid sequence of SEQ ID NO: 69, and a light chain variable region L-CDR3 amino acid sequence of SEQ ID NO: 70.

In certain embodiments of the first aspect the anti-CECAM6 antibody mentioned supra comprises: a heavy chain variable region (VH) comprising the amino acid sequence of SEQ ID NO: 63, and a light chain variable region (VL) comprising the amino acid sequence of SEQ ID NO: 67.

In certain embodiments of the first aspect the anti-CECAM6 antibody mentioned supra comprises: a heavy chain (HC) comprising the amino acid sequence of SEQ ID NO: 71, and a light chain (LC) comprising the amino acid sequence of SEQ ID NO: 72.

In certain embodiments the anti-CECAM6 antibody comprises a heavy chain (HC) comprising the amino acid sequence of SEQ ID NO: 71, and a light chain (LC) comprising the amino acid sequence of SEQ ID NO: 72.

In certain preferred embodiments of the first aspect the anti-CECAM6 antibody mentioned supra is an isolated antibody.

In certain preferred embodiments of the first aspect the anti-CECAM6 antibody mentioned supra is a monoclonal antibody.

In certain preferred embodiments of the first aspect the anti-CECAM6 antibody mentioned supra is a human or humanized antibody.

In certain embodiments of the first aspect the anti-CECAM6 antibody mentioned supra binds to CEACAM6 comprising the amino acid sequence of SEQ ID NO: 75.

In certain embodiments of the first aspect the anti-CECAM6 antibody mentioned supra binds to CEACAM6 domain 1 comprising the amino acids 35-142 of SEQ-ID NO: 75.

### Antibody Generation

It is a further aspect of the invention to provide a method to generate the antibodies of the first aspect. A detailed description how to provide antibodies having certain binding properties is disclosed in WO 2016/150899 A2.

An antibody of the invention may be derived from a recombinant antibody library that is based on amino acid sequences that have been isolated from the antibodies of a large number of healthy volunteers e.g. using the n-CoDeR® technology the fully human CDRs are recombined into new antibody molecules (Carlson & Söderlind, Expert Rev Mol Diagn. 2001 May; 1(1): 102-8). Or alternatively for example antibody libraries as the fully human antibody phage display library described in Hoet R M et al., Nat Biotechnol 2005; 23(3): 344-8) can be used to isolate CEACAM6-specific antibodies. Antibodies or antibody fragments isolated from human antibody libraries are considered human antibodies or human antibody fragments herein.

Human antibodies may be further prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. For example immunization of genetically engineered mice inter alia immunization of hMAb mice (e.g. Velocimmune Mouse® or XENOMOUSE®) may be performed.

Further antibodies may be generated using the hybridoma technology (for example see Köhler and Milstein Nature. 1975 Aug. 7; 256 (5517): 495-7), resulting in for example murine, rat, or rabbit antibodies which can be converted into chimeric or humanized antibodies. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osboum et al., Methods 36:61-68 (2005) and Klimka et al., Br. J. Cancer, 83:252-260 (2000) (describing the “guided selection” approach to FR shuffling).

### Peptide Variants

Antibodies of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to CEACAM6 fall within the scope of the present invention.

A variant can include, for example, an antibody that has at least one altered complementary determining region (CDR) (hyper-variable) and/or framework (FR) (variable) domain/position, vis-à-vis a peptide sequence disclosed herein.

By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected from the sequences provided. The skilled worker can use this to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g. by synthesizing a collection of DNA molecules using trinucleotide mutagenesis (TRIM) technology (Virnekäs B. et al., Nucl. Acids Res. 1994, 22:5600.). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in tums. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

### Antibody-Drug Conjugates (ADC)

The invention also provides antibody-drug conjugates (ADC, immunoconjugates) comprising an anti-CEACAM6 antibody of the first aspect conjugated to one or more cytotoxic agents, such as chemotherapeutic agents or drugs, growth inhibitory agents, toxins (e.g., protein toxins, enzymatically active toxins of bacterial, fungal, plant, human or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP0425235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof; an anthracycline such as daunomycin or doxorubicin; methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alphasarcin, Aleurites fordii proteins, dianthin proteins, Phytolaca americana proteins (P API, P APII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include ²²⁷Th, ²²⁵Ac, ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu. When the radioconjugate is used for detection, it may comprise a radioactive atom for scintigraphic studies, for example Tc99m, or a spin label for nuclear magnetic resonance (NMR) imaging, such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:12 7-131 (1992).

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In a further aspect the invention provides an anti-CEACAM6 antibody of the first aspect conjugated to one or more cytotoxic agents mentioned supra to form an ADC.

### DNA Molecules of the Invention

The present invention also relates to the DNA molecules that encode an antibody of the invention. The DNA sequences used for the antibodies expressed are given for example for TPP-21518 in Table 0 and in the sequence listing. These sequences are optimized in certain cases for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness.

Hybridization stringency, therefore, directly correlates with the structural relationships of two nucleic acid sequences.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent DNA Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized, and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression of Anti-CEACAM6 Antibodies

The present invention further provides recombinant DNA constructs that encode an antibody of the invention. These recombinant constructs of the present invention can be used in connection with a vector, such as a plasmid, phagemid, phage or viral vector, into which a DNA molecule encoding an antibody of the invention or antigen-binding fragment thereof or variant thereof is inserted.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al . . .

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker.

The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic hosts cells are yeasts, insects and insect cells, plants and plant cells, transgenic animals, or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

Therefore, an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore another embodiment of the present invention is the production of the antibodies according to this invention with the host cells of the present invention and purification of these antibodies to at least 95% homogeneity by weight.

### Bacterial Expression

Useful expression vectors for bacterial use are constructed by inserting a DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. Expression of the antibodies may be constitutive or regulated (e.g. inducible by addition or removal of small molecule inductors such as Tetracyclin in conjunction with Tet system). For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., The recombinant expression vectors can also include origins of replication and selectable markers (see e.g., U.S. Pat. Nos. 4,399,216, 4,634,665 and 5,179,017). Suitable selectable markers include genes that confer resistance to drugs such as G418, puromycin, hygromycin, blasticidin, zeocin/bleomycin or methotrexate or selectable marker that exploit auxotrophies such as Glutamine Synthetase (Bebbington et al., Biotechnology (N Y). 1992 February; 10 (2): 169-75), on a host cell into which the vector has been introduced. For example, the dihydrofolate reductase (DHFR) gene confers resistance to methotrexate, neo gene confers resistance to G418, the bsd gene from Aspergillus terreus confers resistance to blasticidin, puromycin N-acetyl-transferase confers resistance to puromycin, the Sh ble gene product confers resitance to zeocin, and resistance to hygromycin is conferred by the E. coli hygromycin resistance gene (hyg or hph). Selectable markers like DHFR or Glutamine Synthetase are also useful for amplification techniques in conjunction with MTX and MSX.

Transfection of the expression vector into a host cell can be carried out using standard techniques such as electroporation, nucleofection, calcium-phosphate precipitation, lipofection, polycation-based transfection such as polyethylenimine (PEI)-based transfection and DEAE-dextran transfection.

Suitable mammalian host cells for expressing the antibodies, antigen binding fragments thereof or variants thereof provided herein include Chinese Hamster Ovary (CHO cells) such as CHO-K1, CHO-S, CHO-K1SV [including dhfr-CHO cells, described in Urlaub and Chasin, (1980) Proc. Natl. Acad. Sci. USA 77:4216-4220 and Urlaub et al., Cell. 1983 June; 33 (2): 405-12, used with a DHFR selectable marker, e.g., as described in R. J. Kaufman and P. A. Sharp (1982) Mol. Biol. 159:601-621; and other knockout cells exemplified in Fan et al., Biotechnol Bioeng. 2012 April; 109 (4): 1007-15], NSO myeloma cells, COS cells, HEK293 cells, HKB11 cells, BHK21 cells, CAP cells, EB66 cells, and SP2 cells.

Expression might also be transient or semi-stable in expression systems such as HEK293, HEK293T, HEK293-EBNA, HEK293E, HEK293-6E, HEK293-Freestyle, HKB11, Expi293F, 293EBNALT75, CHO Freestyle, CHO—S, CHO-K1, CHO-K1SV, CHOEBNALT85, CHOS-XE, CHO-3E7 or CAP-T cells (for instance Durocher et al., Nucleic Acids Res. 2002 Jan. 15; 30(2):E9).

In some embodiments, the expression vector is designed such that the expressed protein is secreted into the culture medium in which the host cells are grown. The antibodies, antigen binding fragments thereof or variants thereof can be recovered from the culture medium using standard protein purification methods.

### Purification

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from an eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or non-reducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

### Therapeutic Methods

In a further aspect, the present invention relates to therapeutic methods.

Therapeutic methods involve administering to a subject in need of treatment a therapeutically effective amount of an antibody or an antigen-binding fragment thereof or a variant thereof contemplated by the invention. A “therapeutically effective” amount hereby is defined as the amount of an antibody or antigen-binding fragment that is of sufficient quantity to reduce proliferation of CEACAM6 positive cell or to reduce size of a CEACAM6 expressing tumor in a treated area of a subject-either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

It is an embodiment of the invention to provide an antibody or antigen-binding fragment thereof for use as a medicament for the treatment of cancer. In a preferred embodiment the cancer is a tumor and in a highly preferred embodiment the cancer is a solid tumor.

It is an embodiment of the invention to use the antibody or antigen-binding fragment thereof in the manufacture of a medicament for the treatment of a disease.

It is an embodiment of the invention to use the antibody or antigen-binding fragment thereof in the manufacture of a medicament for the treatment of cancer. In a preferred embodiment the cancer is a tumor and in a highly preferred embodiment the cancer is a solid tumor.

The inventive antibodies can be used as a therapeutic or a diagnostic tool in a variety of situations with aberrant CEACAM6-signaling, e.g. cell proliferative disorders such as cancer or fibrotic diseases. Disorders and conditions particularly suitable for treatment with an antibody of the inventions are solid tumors, such as cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypothalmic glioma, cerebellar and cerebral astrocytoma, glioblastoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to transitional cell carcinoma, squamous cell carcinoma, adenocarcinoma, sarcoma and small cell carcinoma.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to squamous cell cancer of the head and neck, laryngeal, hypopharyngeal, nasopharyngeal, oropharyngeal cancer, salivary gland cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

In a preferred embodiment, the antibodies of the invention or antigen-binding fragments thereof are suitable for a therapeutic or diagnostic method for the treatment or diagnosis of a cancer disease comprised in a group consisting of colorectal cancer, non-small-cell lung cancer (NSCLC), small cell lung cancer (SCLC), pancreatic cancer, gastric cancer, breast cancer and multiple myeloma.

The disorders mentioned above have been well characterized in humans, but also exist with a similar etiology in other animals, including mammals, and can be treated by administering pharmaceutical compositions of the present invention.

An antibody of the invention might be co-administered with known medicaments, and in some instances the antibody might itself be modified. For example, an antibody or an antigen-binding fragment thereof or a variant thereof could be conjugated to a cytotoxic agent or radioisotope to potentially further increase efficacy.

Antibodies of the present invention or antigen-binding fragments thereof or variants thereof may be administered as the sole pharmaceutical agent or in combination with one or more additional therapeutic agents where the combination causes no unacceptable adverse effects. This combination therapy includes administration of a single pharmaceutical dosage formulation which contains an antibody of the invention or an antigen-binding fragment thereof or a variants thereof and one or more additional therapeutic agents, as well as administration of an antibody of the invention and each additional therapeutic agent in its own separate pharmaceutical dosage formulation. For example, an antibody of the invention or an antigen-binding fragment thereof or a variant thereof and a therapeutic agent may be administered to the patient together in a single liquid composition, or each agent may be administered in separate dosage formulation.

Where separate dosage formulations are used, an antibody of the invention or an antigen-binding fragment thereof or a variants thereof and one or more additional therapeutic agents may be administered at essentially the same time (e.g., concurrently) or at separately staggered times (e.g., sequentially).

In particular, antibodies of the present invention or antigen-binding fragments thereof or variants thereof may be used in fixed or separate combination with other secondary agents anti-tumor agents such as alkylating agents, anti-metabolites, plant-derived anti-tumor agents, hormonal therapy agents, topoisomerase inhibitors, immunologicals, antibodies, antibody drugs, biological response modifiers, anti-angiogenic compounds, cell therapies, and other anti-tumor drugs including but not limited to camptothecin derivatives, kinase inhibitors, targeted drugs.

In this regard, the following is a non-limiting list of examples of secondary agents that may be used in combination with the antibodies of the present invention:

131l-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123l), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

In addition, the antibodies of the invention may be combined with modalities which cause immunogenic cell death including but not limited to ultraviolet light, oxidizing treatments, heat shock, targeted and untargeted radiotherapy, shikonin, high-hydrostatic pressure, oncolytic viruses, and photodynamic therapy.

In addition, the antibodies of the invention may be combined with agents which cause immunogenic cell death including but not limited to sunitinib, JAK2 inhibitors, anthracyclincs, doxorubicin, mitoxantrone, oxaliplatin, and cyclophosphamide, targeted and untargeted microtubule-destabilizing drugs (like e.g. auristatins and maytansinoids).

The compounds of the present invention may also be employed in cancer treatment in conjunction with radiation therapy and/or surgical intervention.

Furthermore, the antibodies of the invention may be utilized, as such or in compositions, in research and diagnostics, or as analytical reference standards, and the like, which are well known in the art.

In a further aspect the invention provides an anti-CECAM6 antibody of the first aspect for use as a medicament, in particular for use as a medicament for the treatment of cancer. In certain embodiments of this aspect a method is provided for treating cancer associated with the undesired presence of CEACAM6, comprising administering to a subject in need thereof an effective amount of the anti-CECAM6 antibody of the first aspect.

In a further aspect the invention provides an anti-CEACAM6 antibody of the first aspect for use in simultaneous, separate, or sequential combination with an anti-PD-1 antibody or an anti-PD-L1 antibody in the treatment of cancer.

In certain embodiments the anti-PD-1 antibody is nivolumab, or pembrolizumab, and the anti-PD-L1 antibody is atezolizumab, avelumab, or durvalumab. In certain embodiments of this aspect a method of treating cancer is provided comprising administering to a patient in need thereof an effective amount of an anti-CEACAM6 antibody of the first aspect in simultaneous, separate, or sequential combination with an anti-PD-1 antibody or an anti-PD-L1 antibody, preferably the anti-PD-1 antibody is nivolumab, or pembrolizumab, and the anti-PD-L1 antibody is atezolizumab, avelumab, or durvalumab.

In certain embodiments, the anti-PD-1 antibody or an antigen-binding portion thereof is nivolumab or has the same CDR regions as nivolumab. Nivolumab (trade name “OPDIVO”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449). In another embodiment, the anti-PD-1 antibody or fragment thereof cross competes with nivolumab.

In other embodiments, the anti-PD-1 antibody or an antigen-binding portion thereof is pembrolizumab or has the same CDR regions as pembrolizumab. Pembrolizumab (trade name “KEYTRUDA”, also known as lambrolizumab, and MK-3475) is a humanized monoclonal IgG4 antibody directed against human cell surface receptor PD-1. Pembrolizumab is described, for example, in U.S. Pat. No. 8,900,587.

In other embodiments, the anti-PD-1 antibody or an antigen-binding portion thereof is MEDI0608 (formerly AMP-514) or has the same CDR regions as MEDI0608. MEDI0608 is a monoclonal antibody against the PD-1 receptor. MEDI0608 is described, for example, in U.S. Pat. No. 8,609,089, B2.

In other embodiments, the anti-PD-1 antibody or an antigen-binding portion thereof is BGB-A317 or has the same CDR regions as BGB-A317. BGB-A317 is a humanized monoclonal antibody described in U.S. Publ. No. 2015/0079109.

In certain embodiments, the anti-PD-L1 antibody or an antigen-binding portion thereof is atezolizumab or has the same CDR regions as atezolizumab. Atezolizumab (trade name “TECENTRIQ”) also known as MPDL3280A, RG7446) is described in U.S. Pat. No. 8,217,149.

In other embodiments, the anti-PD-L1 antibody or an antigen-binding portion thereof is avelumab or has the same CDR regions as avelumab. Avelumab (trade name “BAVENCIO”) also known as MSB0010718C is described in US 2014/0341917.

In other embodiments, the anti-PD-L1 antibody or an antigen-binding portion thereof is durvalumab or has the same CDR regions as durvalumab. Durvalumab (trade name “IMFINZI”) also known as MEDI4736) is described in U.S. Pat. No. 8,779,108 or US 2014/0356353.

In other embodiments, the anti-PD-L1 antibody or an antigen-binding portion thereof is BMS-936559 or has the same CDR regions as BMS-936559. BMS-936559 (formerly 12A4 or MDX-1105) is a fully human IgG4 monoclonal antibody that targets the PD-1 ligand PD-L1 and is described in U.S. Pat. No. 7,943,743 or WO 2013/173223.

In a further aspect the invention provides an anti-CEACAM6 antibody of the first aspect for use in simultaneous, separate, or sequential combination with an anti-TIM-3 antibody in the treatment of cancer. In certain embodiments the anti-TIM-3 antibody is cobolimab, MBG-453, BMS-986258, Sym-023, LY-3321367 or INCAGN-2390. In certain embodiments of this aspect a method of treating cancer is provided comprising administering to a patient in need thereof an effective amount of the anti-CEACAM6 antibody of the first aspect in simultaneous, separate, or sequential combination with an anti-TIM-3 antibody, preferably the anti-TIM-3 antibody is cobolimab, MBG-453, BMS-986258, Sym-023, LY-3321367 or INCAGN-2390.

In certain embodiments the anti-TIM-3 antibody or an antigen-binding portion thereof is cobolimab (TSR-022, Tesaro), or has the same CDR regions as cobolimab. Cobolimab is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. Cobolimab is described, for example, in WO2016161270 A1 and WO 2018129553 A1. Cobolimab is currently in clinical trials; ClinicalTrials.gov Identifier: NCT02817633 and NCT03680508.

In other embodiments the anti-TIM-3 antibody or an antigen-binding portion thereof is MBG-453 (Novartis) or has the same CDR regions as MBG-453. MBG-453 is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. MBG-453 is described, for example, in WO 2015117002 A1. MBG-453 is registered under CAS No: 2128742-61-8. MBG-453 is currently in clinical trials; ClinicalTrials.gov Identifier: NCT02608268 and NCT03066648.

In other embodiments the anti-TIM-3 antibody or an antigen-binding portion thereof is BMS-986258 (Bristol-Myers Squibb, Five Prime), or has the same CDR regions as BMS-986258.

BMS-986258 is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. BMS-986258 is currently in clinical trials; ClinicalTrials.gov Identifier: NCT03446040. BMS-986258 is described, for example, in WO 2018013818 A2.

In other embodiments the anti-TIM-3 antibody or an antigen-binding portion thereof is Sym-023 (Symphogen), or has the same CDR regions as Sym-023. Sym-023, is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. Sym-023 is currently in clinical trials; ClinicalTrials.gov Identifier: NCT03489343. Sym-023 is described, for example, in WO 2017178493 A1.

In other embodiments the anti-TIM-3 antibody or an antigen-binding portion thereof is LY-3321367 (Eli Lilly), or has the same CDR regions as LY-3321367. LY-3321367 is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. LY-3321367 is currently in clinical trials; ClinicalTrials.gov Identifier: NCT03099109. LY-3321367 is described, for example, in WO 2018039020 A1.

In other embodiments the anti-TIM-3 antibody or an antigen-binding portion thereof is INCAGN-2390 (Agenus), or has the same CDR regions as INCAGN-2390. INCAGN-2390 is a TIM-3 immune checkpoint inhibitor antibody that selectively prevents interaction with some of the known TIM-3 ligands (HMGB1, Galectin-9, Phosphatidylserine (PS), thereby blocking the down-regulation of antitumor T-cell functions. INCAGN-2390 is currently in clinical trials; ClinicalTrials.gov Identifier: NCT03652077. INCAGN-2390 is described, for example, in WO 2017205721 A1.

In other embodiments the anti-TIM-3 antibody or an antigen-binding portion thereof is MAB2365 from R&D Jackson Immunoresearch, or has the same CDR regions as MAB2365. MAB2365 is an rIgG2 antibody.

### Diagnostic Methods

In a further aspect, the present invention relates to diagnostic methods. Anti-CEACAM6 antibodies or antigen-binding fragments thereof can be used for detecting the presence of CEACAM6-expressing tumors. The presence of CEACAM6-containing cells or shed CEACAM6 within various biological samples, including serum, and tissue biopsy specimens, may be detected with anti-CEACAM6 antibodies. In addition, anti-CEACAM6 antibodies may be used in various imaging methodologies such as immunoscintigraphy with a ⁹⁹Tc (or other isotope) conjugated antibody. For example, an imaging protocol similar to the one described using a 111 In conjugated anti-PSMA antibody may be used to detect pancreatic or ovarian carcinomas (Sodee et al., Clin. Nuc. Med. 21:759-766, 1997). Another method of detection that can be used is positron emitting tomography by conjugating the antibodies of the invention with a suitable isotope (see Herzog et al., J. Nucl. Med. 34:2222-2226, 1993).

### Pharmaceutical Compositions and Administration

In a further aspect, the present invention relates to pharmaceutical compositions comprising an anti-CEACAM6 antibody of the first aspect and administration of anti-CEACAM6 antibody of the first aspect. To treat any of the aforementioned disorders, pharmaceutical compositions for use in accordance with the present invention may be formulated in a conventional manner using one or more physiologically acceptable carriers or excipients. An antibody of the invention or antigen-binding fragment thereof can be administered by any suitable means, which can vary, depending on the type of disorder being treated. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the invention or an antigen-binding fragment thereof or a variant thereof might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

An embodiment of the present invention are pharmaceutical compositions which comprise anti-CEACAM6 antibodies of the first aspect or antigen-binding fragments thereof or variants thereof, alone or in combination with at least one other agent, such as a stabilizing compound, which may be administered in any sterile, biocompatible pharmaceutical carrier, including, but not limited to, saline, buffered saline, dextrose, and water. A further embodiment are pharmaceutical compositions comprising a CEACAM6 binding antibody or antigen-binding fragment thereof and a further pharmaceutically active compound that is suitable to treat CEACAM6 related diseases such as cancer. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished often parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine or phosphate or Tris, 0.1%-2% sucrose and/or 2%-7% mannitol at a pH range of 4.5 to 7.5 optionally comprising additional substances like polysorbate that is combined with buffer prior to use.

After pharmaceutical compositions comprising a compound of the invention formulated in an acceptable carrier have been prepared, they can be placed in an appropriate container and labeled for treatment of an indicated condition. For administration of anti-CEACAM6 antibodies or antigen-binding fragment thereof, such labeling would include amount, frequency and method of administration.

### Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

A further preferred embodiment of the invention is:
1. An anti-CECAM6 antibody comprising an IgG1 Fc region lacking the glycans attached to the conserved N-linked site in the CH2 domains of the Fc region, wherein said IgG1 Fc region comprises at least the amino acid substitutions L234A and L235A, as numbered according to the EU index of Kabat.
2. The anti-CECAM6 antibody of embodiment 1, wherein the IgG1 Fc region comprises an amino acid substitution N297A, N297G, or N297Q as numbered according to the EU index of Kabat.
3. An anti-CECAM6 antibody comprising an IgG1 Fc region, wherein said IgG1 Fc region comprises at least the amino acid substitutions N297A, L234A, and L235A as numbered according to the EU index of Kabat.
4. The anti-CECAM6 antibody of any of embodiments 1 to 3, wherein said antibody competes for CEACAM6 binding with an antibody comprising a heavy chain variable region (VH) comprising the amino acid sequence of Seq ID No: 63 and a light chain variable region (VL) comprising the amino acid sequence of Seq ID No: 67.
5. The anti-CECAM6 antibody of any of embodiments 1 to 4, wherein said antibody comprises:
   a. a heavy chain variable region H-CDR1 comprising the amino acid sequence of SEQ ID NO: 64,
   b. a heavy chain variable region H-CDR2 comprising the amino acid sequence of SEQ ID NO: 65,
   c. a heavy chain variable region H-CDR3 comprising the amino acid sequence of SEQ ID NO: 66,
   d. a light chain variable region L-CDR1 comprising the amino acid sequence of SEQ ID NO: 68,
   e. a light chain variable region L-CDR2 comprising the amino acid sequence of SEQ ID NO: 69, and
   f. a light chain variable region L-CDR3 comprising the amino acid sequence of SEQ ID NO: 70.
6. The anti-CECAM6 antibody of any of embodiments 1 to 4, wherein said antibody comprises:
   a. a heavy chain variable region H-CDR1 amino acid sequence of SEQ ID NO: 64,
   b. a heavy chain variable region H-CDR2 amino acid sequence of SEQ ID NO: 65,
   c. a heavy chain variable region H-CDR3 amino acid sequence of SEQ ID NO: 66,
   d. a light chain variable region L-CDR1 amino acid sequence of SEQ ID NO: 68,
   e. a light chain variable region L-CDR2 amino acid sequence of SEQ ID NO: 69, and
   f. a light chain variable region L-CDR3 amino acid sequence of SEQ ID NO: 70.
7. The anti-CECAM6 antibody of any of embodiments 1 to 6, wherein said antibody comprises:
   a. a heavy chain variable region (VH) comprising the amino acid sequence of SEQ ID NO: 63, and
   b. a light chain variable region (VL) comprising the amino acid sequence of SEQ ID NO: 67.
8. The anti-CECAM6 antibody of any of embodiments 1 to 7, wherein said antibody comprises:
   a. a heavy chain (HC) comprising the amino acid sequence of SEQ ID NO: 71, and
   b. a light chain (LC) comprising the amino acid sequence of SEQ ID NO: 72.
9. An anti-CECAM6 antibody, wherein said antibody comprises:
   a. a heavy chain (HC) comprising the amino acid sequence of SEQ ID NO: 71, and
   b. a light chain (LC) comprising the amino acid sequence of SEQ ID NO: 72.
10. An anti-CECAM6 antibody consisting of.
   a. a heavy chain (HC) comprising the amino acid sequence of SEQ ID NO: 71, and
   b. a light chain (LC) comprising the amino acid sequence of SEQ ID NO: 72.
11. The anti-CECAM6 antibody of any of embodiments 1 to 10, wherein said antibody is isolated.
12. The anti-CECAM6 antibody of any of embodiments 1 to 11, wherein said antibody is a monoclonal antibody.
13. The anti-CECAM6 antibody of any of embodiments 1 to 12, wherein said antibody is human or humanized.
14. The anti-CECAM6 antibody of any of embodiments 1 to 13, wherein said antibody specifically binds to CEACAM6 comprising the amino acid sequence of SEQ ID NO: 75.
15. The anti-CECAM6 antibody of any of embodiments 1 to 14, wherein said antibody specifically binds to CEACAM6 domain 1 comprising the amino acids 35-142 of SEQ-ID NO: 75.
16. A nucleic acid that encodes the anti-CECAM6 antibody of any of embodiments 1 to 15.
17. A vector comprising the nucleic acid of embodiment 16.
18. An isolated cell expressing the anti-CECAM6 antibody of any of embodiments 1 to 15 and/or comprising the nucleic acid of embodiment 16 or the vector of embodiment 17.
19. The isolated cell of embodiment 18, wherein said cell is a prokaryotic or a eukaryotic cell.
20. A method of producing the anti-CECAM6 antibody of any of embodiments 1 to 15 comprising culturing of the cell of embodiment 18 and purification of the antibody.
21. An anti-CECAM6 antibody of any of embodiments 1 to 15 for use as a medicament.
22. An anti-CECAM6 antibody of any of embodiments 1 to 15 for use as a medicament for the treatment of cancer.
23. Use of the anti-CECAM6 antibody of any of embodiments 1 to 15 in the manufacture of a medicament for the treatment of a disease.
24. Use of the anti-CECAM6 antibody of any of embodiments 1 to 15 in the manufacture of a medicament for the treatment of a cancer.
25. A method for treating cancer associated with the undesired presence of CECAM6 and/or high prevalence of membrane localized CEACAM6, comprising administering to a subject in need thereof an effective amount of the anti-CECAM6 antibody of any of embodiments 1 to 15.
26. A pharmaceutical composition comprising the anti-CECAM6 antibody of any of embodiments 1 to 15.
27. An anti-CEACAM6 antibody of any of embodiments 1 to 15 for use in simultaneous, separate, or sequential combination with an anti-PD-1 antibody or an anti-PD-L1 antibody in the treatment of cancer.
28. The anti-CEACAM6 antibody for use of embodiment 27, wherein the anti-PD-1 antibody is nivolumab, or pembrolizumab, and the anti-PD-L1 antibody is atezolizumab, avelumab, or durvalumab.
29. A method of treating cancer comprising administering to a patient in need thereof an effective amount of the anti-CEACAM6 antibody of any of embodiments 1 to 15 in simultaneous, separate, or sequential combination with an anti-PD-1 antibody or an anti-PD-L1 antibody.
30. The method of treating cancer of embodiment 29, wherein the anti-PD-1 antibody is nivolumab, or pembrolizumab, and the anti-PD-L1 antibody is atezolizumab, avelumab, or durvalumab.
31. An anti-CEACAM6 antibody of any of embodiments 1 to 15 for use in simultaneous, separate, or sequential combination with an anti-TIM-3 antibody in the treatment of cancer.
32. The anti-CEACAM6 antibody for use of embodiment 31, wherein the anti-TIM-3 antibody is cobolimab, MBG-453, BMS-986258, Sym-023, LY-3321367 or INCAGN-2390.
33. A method of treating cancer comprising administering to a patient in need thereof an effective amount of the anti-CEACAM6 antibody of any of embodiments 1 to 15 in simultaneous, separate, or sequential combination with an anti-TIM-3 antibody.
34. The method of treating cancer of embodiment 32, wherein the anti-TIM-3 antibody is cobolimab, MBG-453, BMS-986258, Sym-023, LY-3321367 or INCAGN-2390.

### EXAMPLES

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

### Example 1: Generation of Antibodies and Antibody Sequences

An overview of protein sequences of antibodies and reference compounds used is provided in Table 1.

TABLE 1
Name, Protein-IDs and SEQ-IDs used in this study

| Name | Protein-ID | Description | SEQ-IDs |
|---|---|---|---|
| Neo201 (human IgG1) | TPP-1173 | based on | SEQ ID NO: 9 & |
| | | US20130189268 | SEQ ID NO: 10 |
| Neo201 (human IgG2) | TPP-3688 | based on | SEQ ID NO: 31 & |
| | | US20130189268 | SEQ ID NO: 32 |
| 9A6 (chimeric hIgG2) | TPP-3470 | Based on | SEQ ID NO: 21 & |
| | | Genovac/Aldevron | SEQ ID NO: 22 |
| | | (GM-0509) | |
| 9A6 (chimeric hIgG1) | TPP-1745 | Based on | Based on/ |
| | | Genovac/Aldevron | variant of |
| | | (GM-0509) | SEQ ID NO: 21 & |
| | | | SEQ ID NO: 22 |
| CEACAM6-Human | TPP-3310 | Based on WO | SEQ ID NO: 19 & |
| IgG2 | | 2016/150899 A2 | SEQ ID NO: 20 |
| CEACAM6-Human | TPP-5468 | | SEQ ID NO: 41 & |
| IgG1 | | | SEQ ID NO: 42 |
| CEACAM6-Human | TPP-10914 | | SEQ ID NO: 51 & |
| IgG1-aglyco | | | SEQ ID NO: 52 |
| CEACAM6-Human | TPP-19919 | | SEQ ID NO: 61 |
| IgG1-LALA | | | SEQ ID NO: 62 |
| CEACAM6-Human | TPP-21518 | | SEQ ID NO: 71 & |
| IgG1-LALAaglyco | | | SEQ ID NO: 72 |
| CEACAM6-Human | APP-1574 | Fab obtained by | |
| Fab (human IgG1- | | papain cleavage of | |
| derived) | | TPP-5468 | |
| CEACAM6-Human | APP-6036 | (Fab)₂ obtained by | |
| (Fab)₂ (human IgG1- | | fabricator cleavage | |
| derived) | | of TPP-5468 | |
| CEACAM6-Human | APP-6849 | (Fab)₂ obtained by | |
| (Fab)₂ (human IgG2- | | fabricator cleavage | |
| derived) | | of TPP-3310 | |
| Control-Human IgG2 | TPP-1238 | Non-binding isotype | |
| | | matched control | |
| Control-Human IgG1 | TPP-754 | Non-binding isotype | |
| | | matched control | |
| Control-Human IgG1- | TPP-2081 | Non-binding isotype | |
| aglyco | | matched control | |
| Control-Human IgG1- | TPP-19924 | Non-binding isotype | |
| LALA | | matched control | |
| Control-Human IgG1- | TPP-21501 | Non-binding isotype | |
| LALAaglyco | | matched control | |
| Control-Human Fab | APP-277 | Non-binding isotype | |
| (human IgG1-derived) | | matched control; | |
| | | obtained by papain | |
| | | cleavage of TPP-754 | |
| Control-Human (Fab)2 | APP-6320 | Non-binding isotype | |
| (human IgG1-derived) | | matched control; | |
| | | obtained by papain | |
| | | cleavage of TPP- | |
| | | 1238 | |

The 9A6 murine IgG1 antibody (GM-0509) was obtained from Genovac and chimerized to human IgG2 or human IgG1. The basis of Neo201 protein sequence as either human IgG1 or human IgG2 was US20130189268. The basis of TPP-3310 CEACAM6-human IgG2 protein sequence was WO 2016/150899 A2. All antibodies were expressed in HEK293 cells using standard transient transfection procedures and purified from the cell culture supernatant via Protein-A and size exclusion chromatography.

Aglycosylated variants (IgG1aglyco) were produced by mutation of Asparagine 297 (numbering according to Eu nomenclature; Edelman et al., Proc Natl Acad Sci USA. 1969 May; 63(1): 78-85; Kabat et al., 1991, Sequences of Proteins of Immunological Interest, 5th Edition. U.S. Department of Health and Human Services, Public Health Service, National Institutes of Health, NIH Publication No. 91-3242) to Alanine. LALA mutations refer to L234A/L235A mutation, whereas LALAaglyco is the triple mutation L234A/L235A/N297A.

Fab and F(ab)₂ proteins were generated by enzymatic cleavage of parental IgGs by papain and fabricator cleavage, respectively. Briefly, immobilized papain (Thermo Fisher Scientific No. 20341) was used for Fab generation according to the manufacturer's recommendations. After cleavage, Fabs were purified using MabSelectSuRe (GE-Healthcare) and size exclusion chromatography using Superdex 200 16/60. Likewise, FabRICATOR (IdeS) (FraglTkit Genovis No. A2-FR2-1000) was used for generation of F(ab)₂ proteins. After cleavage, Fc proteins were removed using Capture Select Fc resin (Thermo Scientific) and F(ab)₂ proteins were further purified by size exclusion chromatography using Superdex 200 16/60.

### Example 2: Clinical Study with TPP-3310 with Occurrence of Neutropenia as Adverse Effect

From patients of 4 dose cohorts receiving 2.5, 5, 10 or 30 mg of the anti-CEACAM6 antibody TPP-3310, respectively, EDTA anti-coagulated peripheral venous blood samples were drawn pre-treatment and at different time points after the start of the infusion. The plasma levels of interleukin 6 (IL-6), interleukin 10 (IL-10) and tumor necrosis factor alpha (TNF-alpha) were determined by Mesoscale ELISA. Myeloperoxidase (MPO) was determined by conventional ELISA.

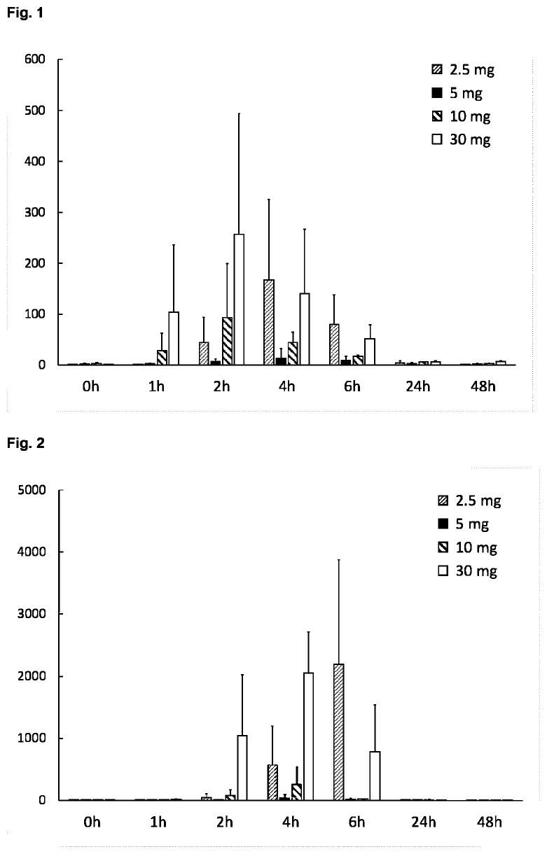

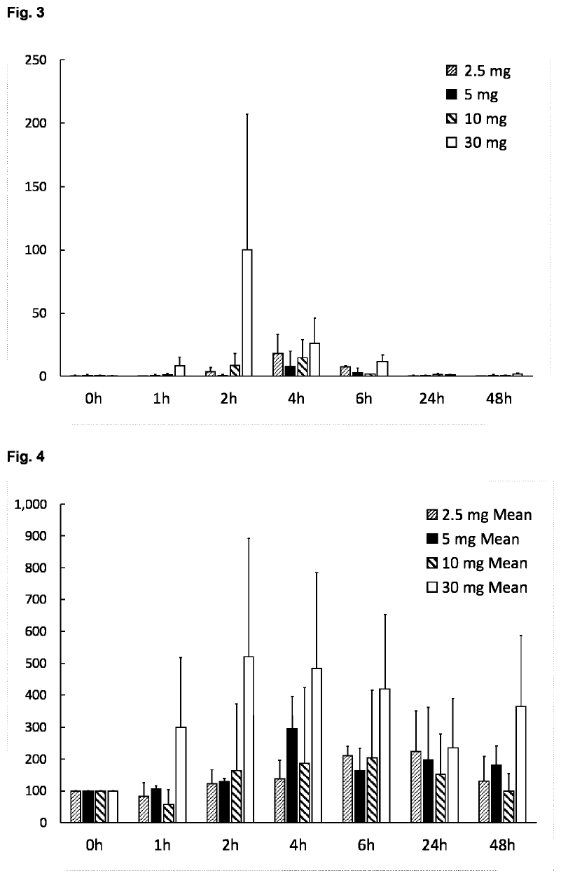

As shown in FIG. 1, FIG. 2, and FIG. 3 a transient systemic mixed inflammatory (TNF-alpha, IL-6)/anti-inflammatory (IL-10) reaction which started after 1-2 hours and was resolved until 24 hours occurred in all dose cohorts. The inflammatory reaction showed a strong inter-patient variability. No dose dependency was observed.

Plasma levels of myeloperoxidase at different time points after start of the i.v. infusion of the anti-CEACAM6 antibody TPP-3310 to cancer patients are shown in FIG. 4.

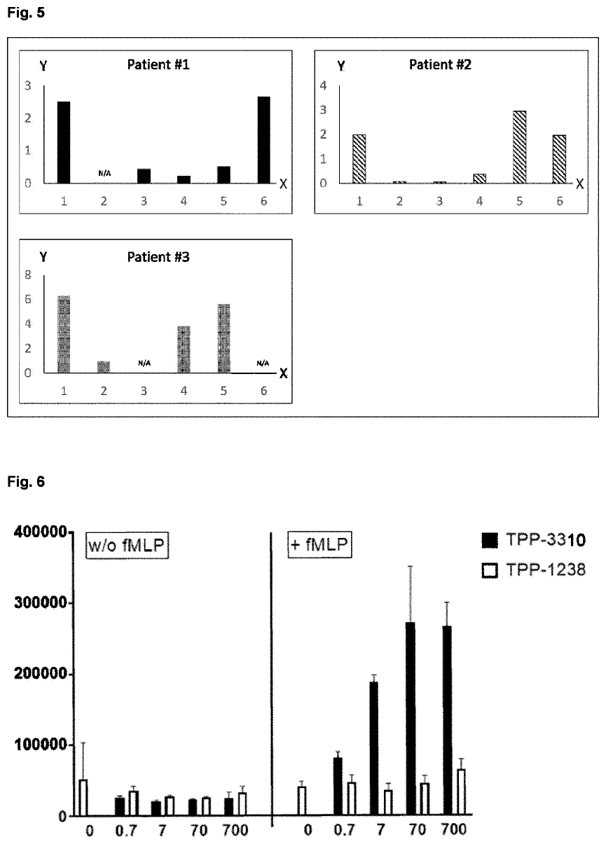

The occurrence of neutropenia (FIG. 5) as an adverse effect in cancer patients treated with low doses of TPP-3310 was astonishing and unexpected.

As shown in FIG. 1, FIG. 2, and FIG. 3, early and transient increases of TNF-alpha, IL-6 and IL-10 were observed and are indicative of an inflammatory event. Strikingly, myeloperoxidase release from neutrophils can be observed with some delay after the first inflammatory event (FIG. 4). This prompted us to scrutinize whether a pre-stimulus (e.g. by inflammatory cytokines) was necessary to detect a detrimental impact of TPP-3310 on neutrophil activation and ultimately depletion that can explain the findings of neutropenia in the clinical trial (FIG. 5).

### Example 3: Assessing Neutrophil Activation by Myeloperoxidase-Release Assay in Whole Blood

It has been known that CEACAM6 is expressed on human neutrophils. Therefore, impact of TPP-3310 on peripheral human whole blood was analyzed even before conducting the clinical trial. In these assays, the amount of myeloperoxidase (MPO) released from neutrophils as activation marker into the supernatant is determined. The test antibody (TPP-3310) was compared to an isotype-matched non-binding control (TPP-1238). No effect has been observed thus far in a variety of donors prior to the clinical trial.

To recapitulate the unexpected findings in clinical studies (especially neutropenia and MPO release), different stimuli in the whole blood assay were tested for their ability to mimic these side effects in vitro. The use of the neutrophil activator fMLP (N-Formylmethionine-leucyl-phenylalanine) proved to be particularly useful, since it allowed to show a detrimental activation effect of anti-CEACAM6 antibody TPP-3310 in this whole blood assay. This effect will otherwise not be picked up under standard assay conditions. The unusual effect was highly reproducible and consistent over a variety of different blood donors.

### Experimental Details

Anticoagulated peripheral human whole blood was incubated with several different anti-CEACAM 6 antibody formats and corresponding isotype control antibodies at titrated concentrations with or without preceding fMLP (N-Formylmethionine-leucyl-phenylalanine) treatment.

After incubation the neutrophil activating capacity of the antibodies was assessed by determining the amount of myeloperoxidase released into the supernatant.

Briefly, whole blood was incubated for 15 min at room temperature in the wells of a microtiter plate in the presence or absence of suboptimal concentration of fMLP (0.01 UM; Sigma Aldrich #F3506). This suboptimal fMLP concentration does not yet lead to measurable release of MPO by the neutrophils. Subsequently, the antibodies were added at the titrated concentrations followed by 2 hours of incubation at 37° C. After incubation the cells were pelleted by centrifugation and supernatant was transferred for a second centrifugation. The supernatant was then stored at −20° C. before until analysis. Analysis was performed using myeloperoxidase human instant ELISA Kit (eBiosciences #BMS2038INST).

As evident from FIG. 6, no impact of anti-CEACAM6 antibody TPP-3310 (human IgG2 format) on neutrophil activation can be detected in whole blood under standard assay conditions (without fMLP). However, upon use of a pre-stimulus (addition of sub-activating fMLP concentrations) a significant and dose-dependent neutrophil activation can be observed, which is reproducible with different human donors. Thus, using these assay conditions the unexpected findings in the clinical study can be translated back to an in vitro assay.

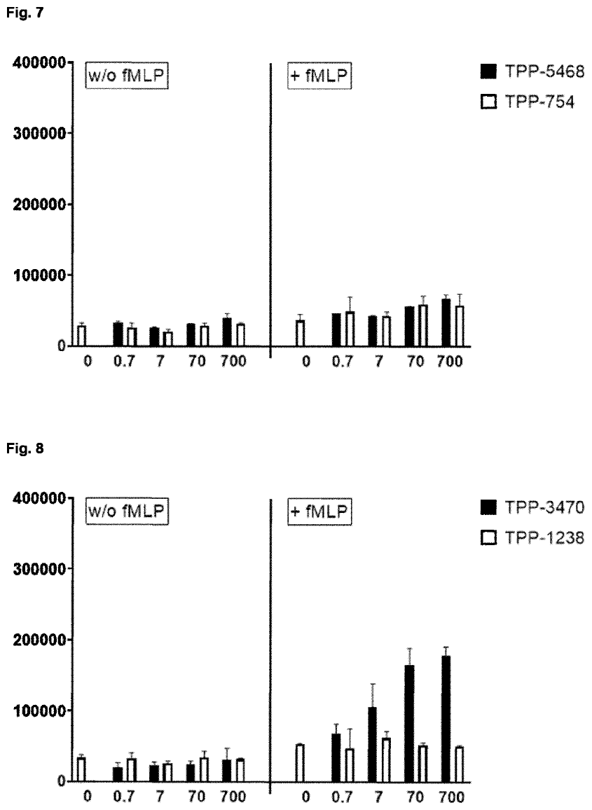

A control was used in these experiments, namely an anti-CECAM6 antibody with the same variable sequences but reformatted into human IgG1 format (TPP-5468). Strikingly, this anti-CEACAM6 human IgG1 format did not result in a neutrophil activation at all, neither without nor with fMLP (FIG. 7). This is highly surprising since human IgG1 antibodies are known to mediate most strongly effector functions through engagement of Fcγ receptors and complement, and thus an even stronger activation was expected.

To further elucidate impact of isotype and epitope, we tested other, unrelated anti-CEACAM6 antibodies with similar and different epitopes. The anti-CEACAM6 antibody 9A6 (TPP-3470 human IgG2) recognized an overlapping epitope with TPP-3310 and competes with binding to membrane-distal N-terminal D1 domain of CEACAM6 (see WO 2016/150899 A2). In contrast, Neo201 (TPP-1173 human IgG1 or TPP-3688 human IgG2) recognizes a different, membrane-proximal epitope on D3 domain of CEACAM6 (also known as B domain; see WO 2016/150899 A2).

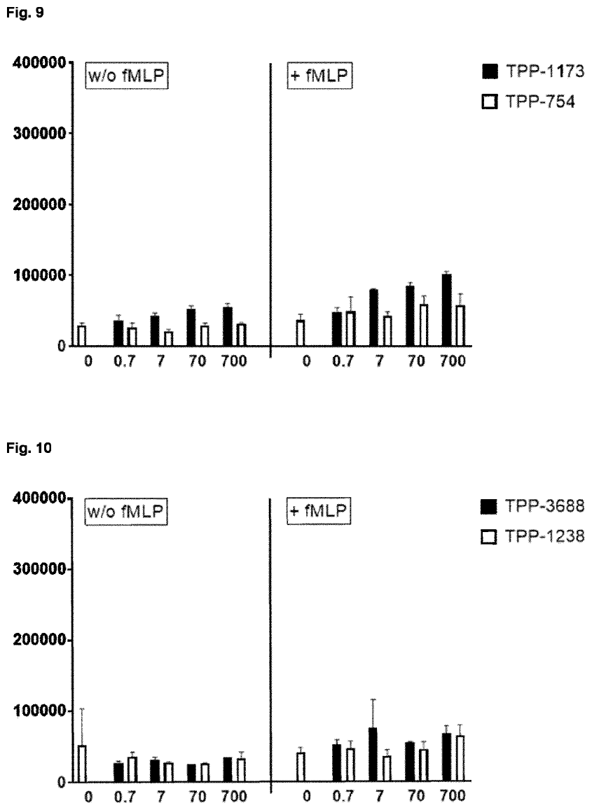

As evident from FIG. 8, FIG. 9, and FIG. 10, the CEACAM6 antibody 9A6, recognizing a very similar epitope to TPP-3310, was able to exert the same neutrophil activation effect. In contrast, the anti-CEACAM6 antibody Neo201, recognizing a different epitope, was unable to activate in neither human IgG1 nor human IgG2 format.

These results implicated both a strong epitope as well as isotype dependency. As mentioned before, difference in effects for anti-CEACAM6 TPP-3310 (human IgG2) and its human IgG1 counterpart (TPP-5468) were unexpected. Thus, we wanted to analyze firstly whether the Fc part of the antibodies played a role and secondly whether Fcγ receptors are involved as well. To this end, monomeric Fab fragments (APP-1574) were tested along with F(ab)₂ dimeric fragments prepared from either IgG1 (APP-6036) or IgG2 (APP-6849). In another series of experiments, the Fcγ receptor blocking antibody AT10 was used, and its impact on neutrophil activation by TPP-3310.

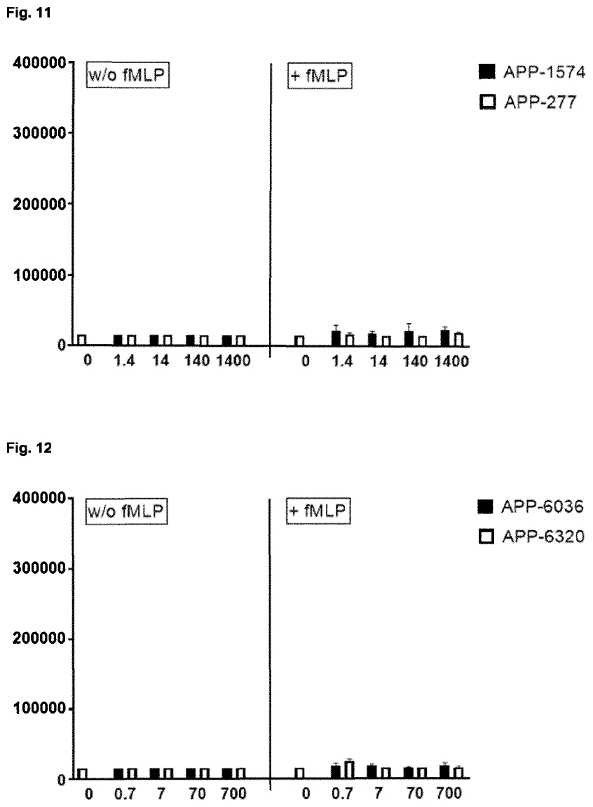

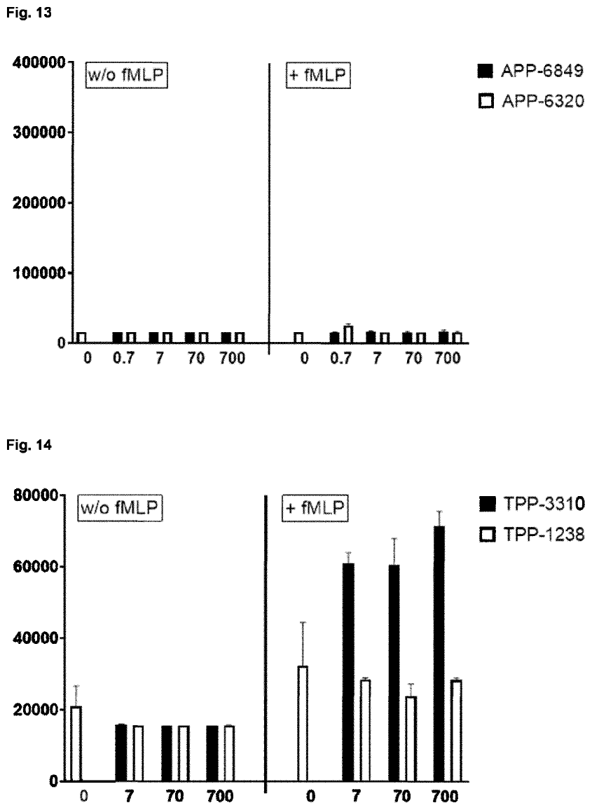

As evident from FIG. 11, FIG. 12, and FIG. 13, neither anti-CEACAM6 Fab fragment APP-1574, nor F(ab)₂ fragments APP-6036 or APP-6849 could mediate an MPO release. This indicates an involvement of the Fc part of the human IgG2 antibody TPP-3310 in MPO release and thus activation of neutrophils.

Next, FcγR blocking experiments were performed by introducing anti-CD32 F(ab′)₂ antibody AT10 (obtained from Biozol) at 1.4 μM concentration prior to addition of the anti-CEACAM 6 antibody TPP-3310. Again, an isotype matched F(ab)₂ fragment served as control.

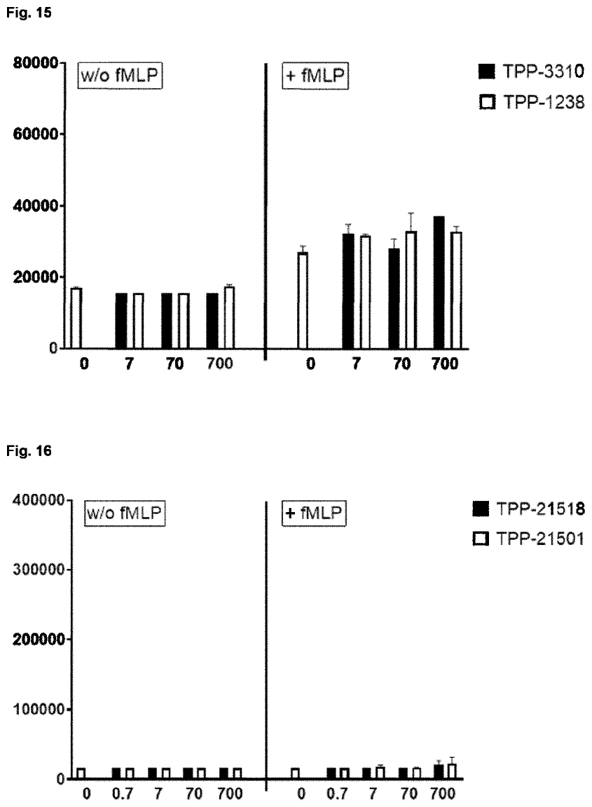

As evident from FIG. 14 and FIG. 15, the MPO release triggered by anti-CEACAM6 antibody TPP-3310 can be inhibited by a CD32 blocking antibody. This demonstrates the dependence of the MPO release effect on engagement of the FcγRII.

In summary, these MPO release experiments demonstrated that anti-CEACAM6 antibody of the human IgG2 isotype format recognizing a membrane distal epitope (TPP-3310 and TPP-3470) are able to activate neutrophils, but only in fMLP pre-stimulated samples, to release MPO. Samples without fMLP pre-stimulation did not lead to MPO release when incubated with the TPP-3310 or TPP-3470. Most notably, the human IgG2 format was strictly required as the same antibody in human IgG1 (TPP-5468) did not exert any effect-despite recognizing the same or similar membrane distal epitope of CEACAM6 as TPP-3310 or TPP-3470.

This MPO release from pre-stimulated samples was not conferred by an anti-CEACAM6 antibody recognizing a more membrane proximal epitope of the CEACAM6 molecule (Neo201 TPP-1173 human IgG1, TPP-3688 human IgG2) irrespective of isotype.

This MPO release from pre-stimulated samples was not conferred by anti-CEACAM6 formats lacking the Fc part of the antibody, like Fab or (Fab) 2 (APP-1574, APP-6036, APP-6849). Furthermore, MPO release experiments conducted under CD32 blocking conditions (by introducing blocking anti-CD32 F(ab′)₂ antibody AT10 prior to addition of the anti-CEACAM6 antibody TPP3310 further demonstrated the dependence of the MPO release effect on engagement of the FcγRII.

In conclusion, these findings implicate a very fine combined dependency of pre-stimulation, epitope and isotype requirement for activation of neutrophils in whole blood. This is completely unpredictable since even the strict dependency on an Fc part as well as the involvement of FcγRII would rather implicate human IgG1 as the more potent molecule, whereas in fact it is completely inactive in this assay. In contrast, the considered to be more silent human IgG2 isotype is in fact the only molecule capable of exerting the neutrophil activation effect.

### Example 4: Determination of Affinity of Different Engineered Anti-CEACAM6 Antibodies to Human Fcγ Receptors Using Surface Plasmon Resonance

In order to analyze the contribution of Fc-Fcγ receptor interactions, the affinities of the interaction (or the absence of which) were determined for different antibody formats, each of them carrying the same variable domains as TPP-3310.

To assess the affinity of different engineered anti-CEACAM6 antibodies, binding assays to human Fcγ receptors were conducted using surface plasmon resonance (SPR).

Binding assays were performed on a Biacore T200 instrument (Cytiva) at 25° C. using assay buffer HBS EP+supplemented with 500 mM NaCl. Fcγ receptors were captured via anti-penta his-tag IgGs (“His capture kit”, Order No. 2895056, Cytiva) covalently amine coupled to a Series S CM5 sensor chip (Cytiva). The amine coupling was carried out according to the manufacturer's instructions using 1-ethyl-3-(3-dimethylaminopropyl) carbodiimide hydrochloride (EDC), N-hydroxysuccinimide (NHS) and ethanolamine HCl, pH 8.5 (“Amine Coupling Kit” BR-1000-50, Cytiva.). Human Fcγ receptor I (R&D Systems, Order No. 1257-FC), Fcγ receptor IIa (R&D Systems, Order No. 1330-CD/F), Fcγ receptor IIb/c (R&D Systems, Order No. 1875-CD), Fcγ receptor IIIa (R&D Systems, Order No. 4325-FC) and Fcγ receptor IIIb (R&D Systems, Order No. 1597-FC) were captured to ˜30 RU.

Anti-CEACAM6 engineered antibodies were used as analytes in a concentration series from 0.04-25 μM in multi cycle kinetics mode. The sensor surface was regenerated with glycine pH 1.5 after each analyte injection. Obtained sensorgrams were double referenced (subtraction of reference flow cell signal and buffer injection) and were fitted to a 1:1 Langmuir binding model to derive steady-state affinity data using the Biacore T200 Evaluation software.

A comparison for anti-CEACAM6 antibody TPP-3310 (human IgG2) and its counterpart as human IgG1 is shown in Table 2. As expected, the interactions between human IgG1 and different FcγR are far stronger than for the considered more silent isotype IgG2. Thus, the results from whole blood MPO release assay from Example 3 are even more puzzling.

TABLE 2
Steady-state affinity values in [M] of anti-CEACAM6
antibody in human IgG1 and human IgG2 format.
Highest concentration used was 16 μM.

| | K_{D} [M] | |
|---|---|---|
| | TPP-5468 | TPP-3310 |
| | “hIgG1” | “IgG2” |
| Fcγ RI | 3.2E−07 | n.b. |
| Fcγ RIIa | 7.3E−06 | n.b. |
| Fcγ RIIb/c | 1.1E−05 | Low binding |
| Fcγ RIIIa | 1.0E−06 | Low binding |
| Fcγ RIIIb | 7.4E−06 | n.b. |

n.b.: no binding
Italics: Approximate value for K_{D}, since even with highest concentration no saturation was reached.

Even if a human IgG1 format such as TPP-5468 would be safe with respect to activation of neutrophils in whole blood as exemplified in Example 3, an IgG1 format is precluded from use in a therapeutic antibody just because of its strong interaction with FcγRs and thus strong and unwanted effector potential such as ADCC, ADCP and CDC activities.

Thus, an IgG1-based format without FcγR interaction and thus without effector function was necessary. To this end, different Fc-engineered variants were tested: an aglycosylated antibody (TPP-10914), an antibody with the “LALA” mutation (TPP-19919) as well as the combination of LALA and aglycosylation mutation (TPP-21518). Results are shown in Table 3.

TABLE 3
Steady-state affinity values in [M] of
engineered anti-CEACAM6 antibodies.

| | K_{D} [M] | | | |
|---|---|---|---|---|
| | | | | TPP-21518 |
| | TPP-5468 | TPP-10914 | TPP-19919 | “LALA |
| | “hIgG1” | “aglyco” | “LALA” | aglyco” |
| Fcγ RI | 1.6E−08 | 1.2E−06 | 3.7E−06 | n.b. |
| Fcγ RIIa | 2.1E−05 | n.b. | n.b. | n.b. |
| Fcγ RIIb/c | >25 μM | n.b. | n.b. | n.b. |
| Fcγ RIIIa | 4.9E−06 | n.b. | >25 μM. | n.b. |
| Fcγ RIIIb | >25 μM | n.b. | n.b | n.b. |

n.b.: no binding
>25 μM: fitted K_{D} value are outside of saturation curve, but clear binding response

Surprisingly, as evident from Table 3, TPP-10914 (“aglyco”) as well as TPP-19919 (“LALA”) both still show binding to Fcγ receptor I as well additionally also a binding response to Fcγ receptor IIIa in case of TPP-19919. Only the combination of the “LALA” together with “aglyco N297A” mutation (TPP-21518) leads to a complete silent isotype that does not show any binding to Fcγ receptors in SPR assay.

### Example 5: Assessing Neutrophil Activation by Myeloperoxidase-Release Assay in Whole Blood

Next, the Fc-engineered silent isotype anti-CEACAM6 antibody TPP-21518 was tested in MPO assay to confirm that also this altered format is unable to exert any unwanted neutrophil activation. The experimental setup was the same as in Example 3.

As can be deduced from FIG. 16, TPP-21518 was unable to trigger an activation in MPO assay with or without fFMLP pre-stimulus.

### Example 6: Assessment of ADCP Potential of Different Antibody Variants

Next, the Fc-engineered silent isotype anti-CEACAM6 antibody TPP-21518 was tested in ADCP assay to confirm that also this altered format is unable to exert any unwanted ADCP activity and thus safe to use for clinical therapy.

Flow cytometry-based readout is employed to track antibody dependent cell phagocytosis (ADCP) of CFSE-labeled neutrophils by primary macrophage. Neutrophils are isolated from freshly drawn whole blood of healthy donors using StemCell EasySep™ Human Neutrophil Isolation Kit (#17957) and used immediately for ADCP. Primary macrophage effector cells are generated from healthy donor peripheral blood mononuclear cells (PBMC). Briefly, CD14+monocyte population is purified from PBMC using the Pan Monocyte Isolation Kit from Miltenyi (#130-096-537) and differentiated in culture for 7-9 days using specific combinations of cytokines and LPS to generate M1, M2a, or M2c macrophage.

Prior to the experiment, neutrophils are pre-treated with 10 nM fMLP for 30 min. ADCP of ˜20,000 CFSE-labeled neutrophils is achieved when co-cultured at a ratio of ˜1:4 with macrophage (˜80,000) in the presence of anti-CEACAM6 antibody for 2 hours at 37° C. The assay is conducted in the presence of 10% normal human serum. Percent ADCP is determined from flow cytometry counts of CFSE positive live macrophage (PI neg, CD206+, CFSE+) over total live macrophage (PI-neg, CD206+).

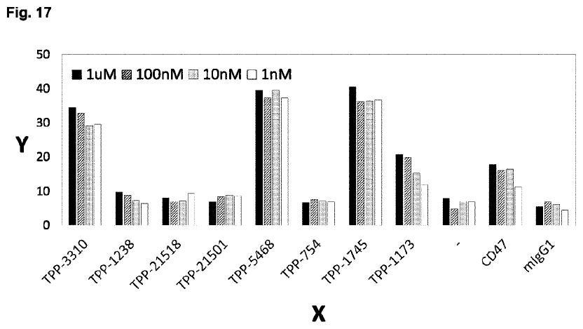

FIG. 17 is representative data (N=4 experiments using unrelated neutrophil and macrophage donors) of the silenced ADCP activity of the human IgG1-LALAaglyco version of anti-CEACAM6 (TPP-21518) relative to unmodified IgG1 (TPP-5468) and IgG2 anti-CEACAM6 (TPP-3310). Absence of phagocytosis with non-binding isotype control antibodies indicates anti-CEACAM6 dependent phagocytosis. Anti-huCD47 positive control mouse antibody (clone B6H12) which blocks the “do not eat me” signal confirms phagocytic activity of macrophage preps and susceptibility of neutrophils for phagocytosis.

### Example 7: Assessment of Different Antibody Variants in T Cell Potency Assay

Next, the Fc-engineered silent isotype anti-CEACAM6 antibody TPP-21518 was tested in T cell potency assay to confirm that also this altered format is still capable to mediate the wanted pharmacological effect and thus suitable to use for clinical therapy.

### In Vitro Pharmacological Potency of Anti-CEACAM6 Antibodies on IL2 Secretion of Survivin Peptide Specific T Cells

Tumor-antigen specific T cells were generated by a procedure described in Brackertz et al (Brackertz et al., Blood Cancer J. 2011 March; 1(3): e1). Briefly, survivin specific CD8+ T cells were isolated from peripheral mononuclear cells via CD8-specific magnetic-activated cell sorting. The isolated HLA-A2 CD8+ T cells were stimulated with HLA-A2 dendritic cells loaded with 10 g of Survivin epitope (ELTLGEFLKL). After stimulation, the proliferating T cells were stained with HLA-A2/Survivin multimers (A*02:0 1 39 1 LMLGEFLKL Survivin 96-1 04 labeled with APC, Prolmmune Limited, #F391-4A-E), FACS sorted and cloned by limiting dilution in 96-well plates. The T cell clone expansion was performed by culturing 2×10⁸ T cell clones and feeder cells composed of 5×10⁷ irradiated PBMCs (30 Gy) and 1×10⁷ irradiated (100-150 Gy) LCL, as described in Brackertz et al., Blood Cancer J. 2011 March; 1(3):e1 in 40 ml of RPMI-1 640 medium with glutamine (Sigma-Aldrich), 10% human serum (Human AB serum, Valley Biomedical, Inc, #HP1 022), 1% Penicillin/Streptomycin (Life Technologies) at 37° C. and 5% CO2. The expansion occurred in the presence of 50 U/ml IL-2 (Proleukin, Novartis, #1003780), 2.5 ng/ml IL-1 5 (mIL-1 5-CF R&D #247_IL-025/CF) and 30 ng/ml anti-human CD3 antibody (OKT3 eBiosciences 16-0037-85) for 14 days. The HCC2935 human lung adenocarcinoma line was cultured in RPMI-1640 (Sigma-Aldrich) with 10% FCS (FBS Superior, Biochrom) and 1% Penicillin/Streptomycin at 37° C. and 5% CO2.

To analyze the modulatory activity of the anti-CEACAM6 antibodies on the immunosuppressive function of CEACAM6 in vitro, the survivin-peptide specific CD8+T cell clone was co-cultivated together with the CEACAM6, lung adenocarcinoma cell line HCC2935. IFN-gamma secretion was used as readout for T cell activity. IFN-gamma was measured in the supernatant IFN-gamma ELISA. For the co-culture, HCC2936 tumor cells were detached non-enzymatically using PBS-EDTA for 5 min. centrifuged, washed and counted. 40,000 HCC2936 target cells were seeded directly in triplicates to IFN-gamma U-96-Well ELISA plates. In the meantime, survivin-peptide specific T cells were harvested, washed with X-Vivo-20 and seeded at 80,000 cells per well. IgG1 LALAaglyco anti-CEACAM6 antibody was added to the well at a final concentration of 0.03-7.5 ug/ml in order to calculate the EC50. The co-culture of tumor cells, anti-CEACAM6 antibodies and T cells was incubated for 24 h at 37° C. IFN-gamma-ELISA (BD human IFN-gamma ELISA Set #5551 42) were developed according to the manufacturer's instructions. Optical density for ELISA plates was measured with a Tecan Infinite M200 plate reader. Co-culture of HCC2936 tumor cells with survivin-peptide specific CD8+ T cells in the presence of anti-CEACAM6 antibodies resulted in a statistically significant increase of IFN-gamma production by the T cells compared to the samples treated with isotype-matched control antibody. The EC50 of the IgG1 LALA aglyco anti-CEACAM6 antibody TPP-21518 in this assay was 0.55 μg/ml

### In Vitro Pharmacological Effect of Anti-CEACAM6 Antibodies on IFNgamma Secretion of Polyclonal Tumor Infiltrating T Cells

Pancreatic cancer tumor infiltrating lymphocyte cell lines (TILs) were isolated from fresh primary culture of tumor tissue from surgery. In brief, fresh primary tissue material was cut into small pieces and cultured in small dishes in X-Vivo-15 medium (Lonza) containing 2% human serum albumin, 2.5 ug/ml Fungizone, 20 μg/ml Gentamycin, 1% Penicillin/Streptomycin with 6000 IU/IL-2 for 10-18 days. Afterwards, cells from the supernatant were harvested and either frozen or used directly for a “rapid expansion protocol” (REP). For rapid expansion of TILs, frozen TILs were gently thawed and cultured with 0.6*106 cells/ml for 1 day in Complete Lymphocyte Medium CLM RPMI-1640 (Life Technologies #21875034), 10% human AB Serum (MILAN Analytica #000083), 1% Penicillin/Streptomycin (Life Technologies #15140122), 1% ml HEPES (Life Technolgies #15630056), 0.01% β-mercaptoethanol [stock 50 mM] (Life Technologies #31350010)) with 6000 IU/ml IL-2. TILs were harvested and expanded at a 1:100 ratio with 60 Gy irradiated feeder PBMCs from 3 different donors in 400 ml REP medium (50% CLM mixed with 50% AIM-V serum free medium (Gibco #12055091) containing 3000 IU/ml IL-2 and 30 ng/ml OKT-3 antibody (eBioscience #16-0037-85)) in G-REX-100 Flasks (Wilson Wolf #80500S). Cells were cultured and split as described in Jin et al., J Immunother. 2012 April; 35 (3): 283-92. After 14 days cells were harvested and frozen in aliquots. Prior to co-culture cytotoxicity assays, individual aliquots of TILs were gently thawed and cultured with 0.6×10⁶ cells/ml for 2 days in CLM containing 6000 IU/ml IL-2 and 1 day in CLM without IL-2.

For the co-culture, HCC2935 tumor cells were detached non-enzymatically using PBS-EDTA for 5 min, centrifuged, washed and counted. 25,000 HCC2936 target cells were seeded directly in triplicates to U-96-Well ELISA plates. In the meantime, TILs cells were thawed, washed with X-Vivo-20 and seeded at 50,000 cells per well. IgG1 LALA aglyco anti-CEACAM6 antibody was added to the co-culture of tumor cells, and T cells. Bispecific antibody anti-CD3× anti-EPCAM IgG (0.25 ng/ml) (Marme et al., Int J Cancer. 2002 Sep. 10; 101 (2): 183-9; Salnikov et al., J Cell Mol Med. 2009 September; 13 (9B): 4023-33) was added to the co-culture to allow for HLA-independent T cell mediated tumor cell killing were also added to increase the increase the recognition of tumor cells by TILs. The co-culture was incubated for 24 h at 37° C. IFN-gamma-ELISA (BD human IFN-gamma ELISA Set #5551 42) were developed according to the manufacturer's instructions. Optical density for ELISA plates was measured with a Tecan Infinite M200 plate reader. Co-culture of HCC2936 tumor cells with TILs CD8+ T cells in the presence of anti-CEACAM6 antibodies resulted in a statistically significant increase of IFN-gamma production by the T cells compared to the samples treated with isotype-matched control antibody. The EC50 of the IgG1 LALAaglyco anti-CEACAM6 antibody TPP-21518 in this assay was 0.5 μg/ml

### In Vitro Pharmacological Effect of Anti-CEACAM6 Antibodies on Tumor Cell Killing Assay Using Tumor Infiltrate CD8+ T Cells.

T cell mediated cytotoxity of HCC2935 tumor cells was analyzed in an impedance-based cytotoxicity assay (xCELLigence) system. In this system cytotoxicity is measured directly and continuously over a long time period of around 100 h (real time). Adherent tumor cells are attached to microelectrodes at the bottom of a 96-Well E-plate (E-Plate VIEW 96 PET; ACEA Biosciences #ID: H000568) which changes the electrical impedance of these electrodes. This is monitored as an increase of the dimensionless “cell index”. After adherence of the tumor cells (24 h) antibodies and T cells are added to the wells which, if T cells exert cytotoxic activity, results in lysis of the tumor cells and detachment from the electrodes. This detachment changes the impedance of the wells and is measured as a decrease of the “cell index” or “normalized cell index” which is the “cell index” normalized to the time point of T cell addition. The T cells alone do not affect the electrical impedance of the electrodes and thus only the cytolysis of the tumor cells is measured. (Peper et al. J Immunol Methods. 2014 March: 405: 192-8).

The effect of the CEACAM6 antibodies on the cytolytic activity of patient-derived TILs cells of a pancreatic cancer was tested. Therefore, 10,000 cells of the CEACAM6 positive lung cancer cell line HCC2935 was added to 96-well plates and cultivated for 24 h. Then, TILs were added at different ratios in the presence of the CEACAM6 antibody (0.03-7.5 μg/ml) and of a bispecific antibody anti-CD3× anti-EPCAM IgG (0.25 ng/ml) (Marme et al., Int J Cancer. 2002 Sep. 10; 101 (2): 183-9; Salnikov et al., J Cell Mol Med. 2009 September; 13(9B): 4023-33) to allow for HLA-independent T cell mediated tumor cell killing. In the presence of the anti-CEACAM6 antibodies we observed a significant cytolytic kill of the target cell line HCC2935. In an additional experiment it could be demonstrated that the effect of the CEACAM6 antibody TPP-21518 is dose dependent and an EC₅₀ value of 0.43 μg/ml was determined for the 100 h timepoint.

All examples were carried out using standard techniques, which are well known and routine to those of skill in the art, except where otherwise described in detail.

Results are shown in Table 4.

TABLE 4
Overview EC50 values obtained for TPP-21518 and
TPP-3310 in different cellular potency assay systems

| | EC50 (μg/ml) | EC50 (μg/ml) |
|---|---|---|
| Functional experiment | TPP-21518 | TPP-3310 |
| survivin specific T cells IFNgamma | 0.55 | 0.5 |
| production | | |
| TILs IFNgamma production | 0.5 | 0.7 |
| TILs mediated HCC2935 killing | 0.43 | 0.4 |

In summary, these experiments show that the CEACAM6 antibody TPP-21518 of the invention has the potential to effectively block the immunosuppressive receptor CEACAM6 and improve the cytotoxic efficacy not only of model T cells but also of patient-derived Tumor infiltrating lymphocytes against CEACAM6 positive tumor cells. In that sense, TPP-21518 has a similar potency as its human IgG2 counterpart TPP-3310 and is thus suitable to use for clinical therapy.

1. An anti-CECAM6 antibody comprising an IgG1 Fc region lacking the glycans attached to the conserved N-linked site in the CH2 domains of the Fc region, wherein said IgG1 Fc region comprises at least the amino acid substitutions L234A and L235A, as numbered according to the EU index of Kabat.

2. The anti-CECAM6 antibody of claim 1, wherein the IgG1 Fc region comprises an amino acid substitution N297A, N297G, or N297Q as numbered according to the EU index of Kabat.

3. An anti-CECAM6 antibody comprising an IgG1 Fc region, wherein said IgG1 Fc region comprises at least the amino acid substitutions N297A, L234A, and L235A as numbered according to the EU index of Kabat.

4. The anti-CECAM6 antibody of claim 1, wherein said antibody competes for CEACAM6 binding with an antibody comprising a heavy chain variable region (VH) comprising the amino acid sequence of Seq ID No: 63 and a light chain variable region (VL) comprising the amino acid sequence of Seq ID No: 67.

5. The anti-CECAM6 antibody of claim 1, wherein said antibody comprises:
a. a heavy chain variable region H-CDR1 comprising the amino acid sequence of SEQ ID NO: 64,
b. a heavy chain variable region H-CDR2 comprising the amino acid sequence of SEQ ID NO: 65,
c. a heavy chain variable region H-CDR3 comprising the amino acid sequence of SEQ ID NO: 66,
d. a light chain variable region L-CDR1 comprising the amino acid sequence of SEQ ID NO: 68,
e. a light chain variable region L-CDR2 comprising the amino acid sequence of SEQ ID NO: 69, and
f. a light chain variable region L-CDR3 comprising the amino acid sequence of SEQ ID NO: 70.

6. The anti-CECAM6 antibody of claim 1, wherein said antibody comprises:
a. a heavy chain variable region H-CDR1 amino acid sequence of SEQ ID NO: 64,
b. a heavy chain variable region H-CDR2 amino acid sequence of SEQ ID NO: 65,
c. a heavy chain variable region H-CDR3 amino acid sequence of SEQ ID NO: 66,
d. a light chain variable region L-CDR1 amino acid sequence of SEQ ID NO: 68,
e. a light chain variable region L-CDR2 amino acid sequence of SEQ ID NO: 69, and
f. a light chain variable region L-CDR3 amino acid sequence of SEQ ID NO: 70.

7. The anti-CECAM6 antibody of claim 1, wherein said antibody comprises:
a. a heavy chain variable region (VH) comprising the amino acid sequence of SEQ ID NO: 63, and
b. a light chain variable region (VL) comprising the amino acid sequence of SEQ ID NO: 67.

8. The anti-CECAM6 antibody of claim 1, wherein said antibody comprises:
a. a heavy chain (HC) comprising the amino acid sequence of SEQ ID NO: 71, and
b. a light chain (LC) comprising the amino acid sequence of SEQ ID NO: 72.

9. An anti-CECAM6 antibody, wherein said antibody comprises:
a. a heavy chain (HC) comprising the amino acid sequence of SEQ ID NO: 71, and
b. a light chain (LC) comprising the amino acid sequence of SEQ ID NO: 72.

10. The anti-CECAM6 antibody of claim 9 consisting of:
a. a heavy chain (HC) comprising the amino acid sequence of SEQ ID NO: 71, and
b. a light chain (LC) comprising the amino acid sequence of SEQ ID NO: 72.

11. The anti-CECAM6 antibody of claim 1, wherein said antibody is isolated.

12. The anti-CECAM6 antibody of claim 1, wherein said antibody is a monoclonal antibody.

13. The anti-CECAM6 antibody of claim 1, wherein said antibody is human or humanized.

14. (canceled)

15. The anti-CECAM6 antibody of claim 1, wherein said antibody specifically binds to CEACAM6 domain 1 comprising the amino acids 35-142 of SEQ-ID NO: 75.

16. A nucleic acid that encodes the anti-CECAM6 antibody claim 1.

17. (canceled)

18. (canceled)

19. (canceled)

20. (canceled)

21. (canceled)

22. (canceled)

23. (canceled)

24. (canceled)

25. A method for treating cancer associated with the undesired presence of CECAM6 and/or high prevalence of membrane localized CEACAM6, comprising administering to a subject in need thereof an effective amount of the anti-CECAM6 antibody of claim 1.

26. (canceled)

27. (canceled)

28. (canceled)

29. The method of treating cancer of claim 25, comprising administering to said subject an effective amount of the anti-CEACAM6 antibody in simultaneous, separate, or sequential combination with an anti-PD-1 antibody or an anti-PD-L1 antibody.

30. The method of treating cancer of claim 29, wherein the anti-PD-1 antibody is nivolumab, or pembrolizumab, and the anti-PD-L1 antibody is atezolizumab, avelumab, or durvalumab.

31. (canceled)

32. (canceled)

33. The method of treating cancer of claim 25, comprising administering to said subject an effective amount of the anti-CEACAM6 antibody in simultaneous, separate, or sequential combination with an anti-TIM-3 antibody.

34. The method of treating cancer of claim 33, wherein the anti-TIM-3 antibody is cobolimab, MBG-453, BMS-986258, Sym-023, LY-3321367 or INCAGN-2390.


SEQUENCE LISTING (75 sequences)

SEQ ID NO: 1 - protein, 121 aa, synthetic construct
  REGION 1..121: note = Antibody sequence
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 2 - protein, 5 aa, synthetic construct
  REGION 1..5: note = Antibody sequence
DYAMH

SEQ ID NO: 3 - protein, 17 aa, synthetic construct
  REGION 1..17: note = Antibody sequence
LISTYSGDTKYNQNFQG

SEQ ID NO: 4 - protein, 12 aa, synthetic construct
  REGION 1..12: note = Antibody sequence
GDYSGSRYWFAY

SEQ ID NO: 5 - protein, 107 aa, synthetic construct
  REGION 1..107: note = Antibody sequence
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIK

SEQ ID NO: 6 - protein, 11 aa, synthetic construct
  REGION 1..11: note = Antibody sequence
QASENIYGALN

SEQ ID NO: 7 - protein, 7 aa, synthetic construct
  REGION 1..7: note = Antibody sequence
GASNLAT

SEQ ID NO: 8 - protein, 9 aa, synthetic construct
  REGION 1..9: note = Antibody sequence
QQVLSSPYT

SEQ ID NO: 9 - protein, 450 aa, synthetic construct
  REGION 1..450: note = Antibody sequence
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
  REGION 1..214: note = Antibody sequence
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 118 aa, synthetic construct
  REGION 1..118: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSS

SEQ ID NO: 12 - protein, 7 aa, synthetic construct
  REGION 1..7: note = Antibody sequence
TYGIGVG

SEQ ID NO: 13 - protein, 16 aa, synthetic construct
  REGION 1..16: note = Antibody sequence
HIWWNDNKYYSTSLKT

SEQ ID NO: 14 - protein, 8 aa, synthetic construct
  REGION 1..8: note = Antibody sequence
ISLPYFDY

SEQ ID NO: 15 - protein, 107 aa, synthetic construct
  REGION 1..107: note = Antibody sequence
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIK

SEQ ID NO: 16 - protein, 11 aa, synthetic construct
  REGION 1..11: note = Antibody sequence
KASQNVGTAVA

SEQ ID NO: 17 - protein, 7 aa, synthetic construct
  REGION 1..7: note = Antibody sequence
SASNRYT

SEQ ID NO: 18 - protein, 9 aa, synthetic construct
  REGION 1..9: note = Antibody sequence
QQYSSYPLT

SEQ ID NO: 19 - protein, 443 aa, synthetic construct
  REGION 1..443: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 20 - protein, 214 aa, synthetic construct
  REGION 1..214: note = Antibody sequence
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 443 aa, synthetic construct
  REGION 1..443: note = Antibody sequence
QVTLKESGPGILQPSQTLTLTCSFSGFSLSTSGLSVGWIRQPSGKGLEWLAHIWWNDDKYYNPALKSRLTISKVTSNNQVFLKIASVVTADTATYYCARIPRGAMDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 22 - protein, 214 aa, synthetic construct
  REGION 1..214: note = Antibody sequence
DIVMTQSQKFMSTSVGDRVSVTCKASQNVYTNVAWYQQKPGQSPKALIYSASYRYSGVPDRFTGSGSGTDFTLTITNVQSEDLAEYFCQQYNSYPLTFGSGTKLEIKRADAAPTVSIFPPSSEQLTSGGASVVCFLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 121 aa, synthetic construct; identical to SEQ ID NO: 1
  REGION 1..121: note = Antibody sequence

SEQ ID NO: 24 - protein, 5 aa, synthetic construct
  REGION 1..5: note = Antibody sequence
DYAMH

SEQ ID NO: 25 - protein, 17 aa, synthetic construct
  REGION 1..17: note = Antibody sequence
LISTYSGDTKYNQNFQG

SEQ ID NO: 26 - protein, 12 aa, synthetic construct
  REGION 1..12: note = Antibody sequence
GDYSGSRYWFAY

SEQ ID NO: 27 - protein, 107 aa, synthetic construct; identical to SEQ ID NO: 5
  REGION 1..107: note = Antibody sequence

SEQ ID NO: 28 - protein, 11 aa, synthetic construct
  REGION 1..11: note = Antibody sequence
QASENIYGALN

SEQ ID NO: 29 - protein, 7 aa, synthetic construct
  REGION 1..7: note = Antibody sequence
GASNLAT

SEQ ID NO: 30 - protein, 9 aa, synthetic construct
  REGION 1..9: note = Antibody sequence
QQVLSSPYT

SEQ ID NO: 31 - protein, 446 aa, synthetic construct
  REGION 1..446: note = Antibody sequence
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 32 - protein, 214 aa, synthetic construct; identical to SEQ ID NO: 10
  REGION 1..214: note = Antibody sequence

SEQ ID NO: 33 - protein, 118 aa, synthetic construct; identical to SEQ ID NO: 11
  REGION 1..118: note = Antibody sequence

SEQ ID NO: 34 - protein, 7 aa, synthetic construct
  REGION 1..7: note = Antibody sequence
TYGIGVG

SEQ ID NO: 35 - protein, 16 aa, synthetic construct
  REGION 1..16: note = Antibody sequence
HIWWNDNKYYSTSLKT

SEQ ID NO: 36 - protein, 10 aa, synthetic construct
  REGION 1..10: note = Antibody sequence
ARISLPYFDY

SEQ ID NO: 37 - protein, 107 aa, synthetic construct; identical to SEQ ID NO: 15
  REGION 1..107: note = Antibody sequence

SEQ ID NO: 38 - protein, 11 aa, synthetic construct
  REGION 1..11: note = Antibody sequence
KASQNVGTAVA

SEQ ID NO: 39 - protein, 7 aa, synthetic construct
  REGION 1..7: note = Antibody sequence
SASNRYT

SEQ ID NO: 40 - protein, 9 aa, synthetic construct
  REGION 1..9: note = Antibody sequence
QQYSSYPLT

SEQ ID NO: 41 - protein, 447 aa, synthetic construct
  REGION 1..447: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 214 aa, synthetic construct; identical to SEQ ID NO: 20
  REGION 1..214: note = Antibody sequence

SEQ ID NO: 43 - protein, 118 aa, synthetic construct; identical to SEQ ID NO: 11
  REGION 1..118: note = Antibody sequence

SEQ ID NO: 44 - protein, 7 aa, synthetic construct
  REGION 1..7: note = Antibody sequence
TYGIGVG

SEQ ID NO: 45 - protein, 16 aa, synthetic construct
  REGION 1..16: note = Antibody sequence
HIWWNDNKYYSTSLKT

SEQ ID NO: 46 - protein, 8 aa, synthetic construct
  REGION 1..8: note = Antibody sequence
ISLPYFDY

SEQ ID NO: 47 - protein, 107 aa, synthetic construct; identical to SEQ ID NO: 15
  REGION 1..107: note = Antibody sequence

SEQ ID NO: 48 - protein, 11 aa, synthetic construct
  REGION 1..11: note = Antibody sequence
KASQNVGTAVA

SEQ ID NO: 49 - protein, 7 aa, synthetic construct
  REGION 1..7: note = Antibody sequence
SASNRYT

SEQ ID NO: 50 - protein, 9 aa, synthetic construct
  REGION 1..9: note = Antibody sequence
QQYSSYPLT

SEQ ID NO: 51 - protein, 447 aa, synthetic construct
  REGION 1..447: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 214 aa, synthetic construct; identical to SEQ ID NO: 20
  REGION 1..214: note = Antibody sequence

SEQ ID NO: 53 - protein, 118 aa, synthetic construct; identical to SEQ ID NO: 11
  REGION 1..118: note = Antibody sequence

SEQ ID NO: 54 - protein, 7 aa, synthetic construct
  REGION 1..7: note = Antibody sequence
TYGIGVG

SEQ ID NO: 55 - protein, 16 aa, synthetic construct
  REGION 1..16: note = Antibody sequence
HIWWNDNKYYSTSLKT

SEQ ID NO: 56 - protein, 8 aa, synthetic construct
  REGION 1..8: note = Antibody sequence
ISLPYFDY

SEQ ID NO: 57 - protein, 107 aa, synthetic construct; identical to SEQ ID NO: 15
  REGION 1..107: note = Antibody sequence

SEQ ID NO: 58 - protein, 11 aa, synthetic construct
  REGION 1..11: note = Antibody sequence
KASQNVGTAVA

SEQ ID NO: 59 - protein, 7 aa, synthetic construct
  REGION 1..7: note = Antibody sequence
SASNRYT

SEQ ID NO: 60 - protein, 9 aa, synthetic construct
  REGION 1..9: note = Antibody sequence
QQYSSYPLT

SEQ ID NO: 61 - protein, 448 aa, synthetic construct
  REGION 1..448: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, synthetic construct; identical to SEQ ID NO: 20
  REGION 1..214: note = Antibody sequence

SEQ ID NO: 63 - protein, 118 aa, synthetic construct; identical to SEQ ID NO: 11
  REGION 1..118: note = Antibody sequence

SEQ ID NO: 64 - protein, 7 aa, synthetic construct
  REGION 1..7: note = Antibody sequence
TYGIGVG

SEQ ID NO: 65 - protein, 16 aa, synthetic construct
  REGION 1..16: note = Antibody sequence
HIWWNDNKYYSTSLKT

SEQ ID NO: 66 - protein, 8 aa, synthetic construct
  REGION 1..8: note = Antibody sequence
ISLPYFDY

SEQ ID NO: 67 - protein, 107 aa, synthetic construct; identical to SEQ ID NO: 15
  REGION 1..107: note = Antibody sequence

SEQ ID NO: 68 - protein, 11 aa, synthetic construct
  REGION 1..11: note = Antibody sequence
KASQNVGTAVA

SEQ ID NO: 69 - protein, 7 aa, synthetic construct
  REGION 1..7: note = Antibody sequence
SASNRYT

SEQ ID NO: 70 - protein, 9 aa, synthetic construct
  REGION 1..9: note = Antibody sequence
QQYSSYPLT

SEQ ID NO: 71 - protein, 448 aa, synthetic construct
  REGION 1..448: note = Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 214 aa, synthetic construct; identical to SEQ ID NO: 20
  REGION 1..214: note = Antibody sequence

SEQ ID NO: 73 - DNA, 1344 bp, synthetic construct
  misc_feature 1..1344: note = Antibody sequence
  source 1..1344: mol_type = other DNA
caggtcacactgagagagtccggccctgccctggtgaaacccacccagaccctgaccctgacatgcaccttcagcggcttcagcctgagcacctacggcatcggcgtgggctggatcagacagccccctggcaaggccctggaatggctggcccacatctggtggaacgacaacaagtactacagcaccagcctgaaaacccggctgaccatcagcaaggacaccagcaagaaccaggtggtgctgaccatgaccaacatggaccccgtggacaccgccacctactactgcgcccggatcagcctgccctacttcgactactggggccagggcaccaccctgaccgtgtcctcagccagcacaaagggccctagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacgccagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagtccagatggcagcagggcaacgtgttcagctgcagcgtgatgcacgaggccctgcacaaccactacacccagaagtccctgagcctgtctcctggcaag

SEQ ID NO: 74 - DNA, 642 bp, synthetic construct
  misc_feature 1..642: note = Antibody sequence
  source 1..642: mol_type = other DNA
gacatccagctgacccagagccccagcttcctgagcgccagcgtgggcgacagagtgaccatcacatgcaaggccagccagaacgtgggcaccgccgtggcctggtatcagcagaagcccggcaaggcccccaagctgctgatctacagcgccagcaaccggtacaccggcgtgcccagcagattcagcggcagcggctccggcaccgagttcaccctgaccatcagcagcctgcagcccgaggacttcgccacctactactgccagcagtacagcagctaccccctgaccttcggcggaggcaccaaggtggaaatcaaacgaaccgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 75 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI